(19) United States
(12) Patent Application Publication
(10) Pub. No.: US 2011/0212440 A1
(43) Pub. Date: Sep. 1, 2011

(54) CELL SORTING DEVICE

(75) Inventors: Jean-Louis Viovy, Paris (FR); Laure Saias, Paris (FR); Jacques Goulpeau, Paris (FR); Antoine-Emmanual Saliba, Paris (FR); Laurent Malaquin, Bretigny Sur Orge (FR)

(73) Assignees: CNRS-DAE, Paris (FR); INSTITUT CURIE, Paris (FR); UNIVERSITE PIERRE ET MARIE CURIE, (PARIS VI), PARIS (FR); FLUIGENT, Paris (FR)

(21) Appl. No.: 13/123,448

(22) PCT Filed: Oct. 12, 2009
(86) PCT No.: PCT/IB09/55207

§ 371 Date: May 17, 2011

Related U.S. Application Data
(60) Provisional application No. 61/104,500, filed on Oct. 10, 2008.

(51) Int. Cl. C12Q 1/68; C12N 5/071; C12Q 1/02; G01N 33/53; C12N 13/00; C12M 1/34; C12M 3/00

(52) U.S. Cl. 435/6.1; 435/325; 435/29; 435/7.21; 435/377; 435/173.9; 435/287.1; 435/289.1

(57) ABSTRACT

An integrated microsystem, comprising: a microchannel, a field generator to create a magnetic field in at least one first portion of the microchannel having a direction substantially collinear with the direction of flow in the portion of the microchannel, the magnetic field also presenting a gradient, wherein the microsystem additionally comprises a detection area in fluid connection with the microchannel,

In the last years, progress in medicine has been strongly stimulated by progress in molecular and cell biology. This is for instance the case for cancer. Cancer research benefits from the massive development of genomics, bioinformatics and imaging technologies, and from high throughput tools borrowing from forefront technological progresses in physics, chemistry, and molecular biology. Although recent, these developments have already let to the development of new biomarkers and associated new drugs, with spectacular changes in the outcome for patients, as described e.g. in Kurian, A. W., et al. (2007). J Clin Oncol, 25, 634-41. For instance, this is the case for breast cancer patients positive for the HER2+ surface receptor, who may be treated with specific drugs based on antibodies towards this receptor (e.g. Herceptin).

So far, however, these molecular approaches to cancer treatment are only relevant to a relatively small number of cancers, relapses still exist. At present, one of the limitations of progress is that molecular biomarkers are searched in the tumour as a whole. Recent research strongly suggests that only a small fraction of the whole tumour may bear most of the proliferative and metastatic power.

With current methods, the molecular characteristics of the most dangerous cells may be hidden by those of the tumour as a whole. It is a major challenge for progress in cancer treatment to be able to perform a detailed molecular characterisation of cancer cells subpopulations in order to prescribe the most efficient treatment. The sorting and analysis of tumour cells is thus of high importance for research, for clinical diagnosis, prognosis and treatment selection and follow-up.

Particularly important fields relate to cells that will lead to metastases, i.e. Disseminated Tumour Cells (DTC), present in organs such as Bone Marrow, or Lymph nodes, micrometastases, and Circulating Tumour Cells (CTC). There is thus a very strong need to develop new methods able to detect and characterize such tumour cells. This is a difficult challenge, since these cells may be present in the sample at very low level, as low as one per 100 000 or even one per million.

Other applications where the specific sorting of rare cells would be of high value for research and clinics are circulating foetal cells in the mother's blood, and circulating endothelial cells, for prediction of cardiovascular diseases, and also for the survey of angiogenesis in cancer development and for the prescription and follow up of anti-angiogenic treatments.

In the following, all categories of potential cells of interest, as some non-exhaustive examples were recalled above, will be described under the generic label “COI”, for “Cells Of Interest”.

The most traditional method for the identification of COI is visual cytometry. After centrifugation and resuspension, blood samples are spread on microscope slides, on which the cells are fixed, permeabilized and stained. Then, they are observed under a microscope at high magnification. This technique is very versatile, since multiple labelling protocols may be applied. It also allows a visual observation of the cell's morphology, which remains a very useful discrimination tool, in the hands of experienced anatomopathologists. However, visualisation is extremely time-consuming and requires the expertise of specialist Medical Doctors (MD).

Another method widely used for cell screening is Flow cytometry. Flow cytometry is a highly automated method, and it has gained in the last years a strong discriminatory power, thanks to the development of multi-labelling strategies. However, it is limited in throughput, and involves a high dispersion of quantitative data. This dispersion is not a serious drawback when working with cell populations abundant in the sample, but it is not adapted for rare cells. Typically, this system is reliable for a few hundred cells in each category, but it is not for cells in proportions below typically one per 10 000. Therefore, it cannot be used for typical CTC detection needs

Strategies based on filtration, as recited e.g. in WO 2006/100366, have also been proposed to resolve problems of traditional methods cited above. This approach has the advantage of simplicity, but also has strong limitations. First, it only sorts cells by size, shape or viscoelastic properties, which is not sufficient, e.g. for sorting different tumour cells subpopulations. Second, to filter the quantities of blood necessary for rare cell screening (typically 10 mL), rather large filter are necessary (10 to 50 cm²). Thus the few captured cells are scattered on large areas, making further manipulation and visualisation relatively tedious.

Cells may also be sorted using magnetic particles bearing antibodies to specific surface antigens of the COI. Units are proposed e.g. by companies DYNAL® or MILTENYI® Typically this sorting is performed by mixing the sample with magnetic micro or nanoparticles grafted with specific antibodies for a given surface antigen, incubating under agitation and collecting with a magnet the magnetic particles with the attached cells of interest. This method is simple to operate. However the captured cells must be characterised after capture. If the beads are large (e.g. DYNAL's units), they aggregate with the cells during magnetic sedimentation and make characterisation difficult. The variant using smaller particles proposed by MILTENYI necessitates specific microcolumns to separate the cells but some remain trapped in the column and thus reduce the sensitive yield of this system. All of these methods, in any case, require a lot of manipulation.

To overcome the above limitations, an automated instrument for rare cells sorting was recently commercialised by VERIDEX® under the names “Cell Track™” and CellSearch™. This system first comprises an automated sorter, which automates batch sorting of cancer cells using magnetic particles in blood samples of 7.5 ml. The CellSearch™ system also comprises a semi-automated image analysis system for visual inspection of captured cells. This system simplifies the task of pathologists, by selecting abnormal cell candidates, and presenting them in a library of images.

The VERIDEX® system is less labour-intensive than conventional hand held sorting, but it still suffers from the main drawbacks of magnetic sorting. In particular, it requires the presence of an enormous excess of magnetic carrier with regards to the captured cells in the final sample, and leads to contamination by non-specific cells due to drainage. Moreover, cells are randomly disposed on a slide and may overlap. Thus their automated identification may be perturbed by the high amount of magnetic particles also present on the slide. In addition, cells may be identified only by a fluorescence signature at relatively low resolution, preventing typing cells by their morphological characteristics.

As an alternative, US2007026416 discloses a device for processing a cellular sample, said device comprising a channel comprising a first array of obstacles that form a network of gaps, wherein said obstacles are configured to cause one or more first cells to preferentially make contact with said obstacles, and wherein at least some of said obstacles comprise one or more capture moieties that selectively bind said first cells. Numerous variants of this invention, reciting various modes of implementation, and various potential applications to cancer diagnosis, prenatal diagnosis, and the like, were disclosed by the same group in WO 2006/108087, US2007099207, wo2006108101, US2007196820, US2007026-413, -469, -414, -415, -416, -417; -418; US2007059-716, -680, -774, -719, -718, -781; US2007172903; US2007231851; US2007259424; US2007264675; WO2007/106598; WO2007147018; US2008090239; WO2007147079; WO2008014516; US2008113358, US20080138809.

These systems are able to sort rare cells with a high efficiency, but they also suffer from several drawbacks. First, they require an expensive and delicate micro fabrication step, in order to achieve accurate obstacles with the right cell size. Each microfluidic device has to be functionalized independently, which is costly and involves reproducibility problems. Also, these microsystems have to be rather thick, and high resolution imaging of the captured cells is difficult.

Thus, in spite of numerous and intense efforts, there is not yet a system usable for the sorting and study of analytes, and particularly for the sorting of cells, combining low cost of fabrication, simplicity of fabrication and of use, high automation, high discriminating power, and high sensitivity for the study of rare cells.

It is an object of the invention to provide such a system, and associated methods.

### SUMMARY OF THE INVENTION

Exemplary embodiments of the invention provide a microfluidic device for capturing, sorting, analyzing, typing or cultivating analytes, comprising at least a microchannel comprising at least an active zone, said active zone comprising at least a capture element, and preferably and array of capture elements, wherein the width of said active zone, or the combined width of active zones is larger than their effective length, preferably larger than twice their effective length, more preferably larger than 5 times their effective length.

The width of an active zone is measured perpendicular to flow direction within the microchannel and the effective length of an active zone is measured parallel to said flow direction.

The analytes may be cells or cell aggregates.

In some of its aspects, the invention also provides a microfluidic device for capturing, sorting, analyzing, typing or cultivating analytes from a sample fluid.

the device comprising at least a microchannel comprising at least one active zone, said active zone comprising at least one capture element, and preferably an array of capture elements.

By “sample fluid”, one means a fluid, in which the analytes are contained. Sample fluid may be a body fluid, a fluid extracted from a liquid or solid sample in which analytes are initially present, or an artificial fluid such as a buffer, in which said analytes have been dissolved or suspended.

In the following description, the term“Nucleic acid” designates not only natural nucleic acids, e.g. DNA and RNA, but also artificial or modified nucleic acids, such as, as a non exhaustive list, PNA, LNA, thiolated nucleic acids, and the like. It can designate, notably, genomic nucleic acids, ribosomal nucleic acids, mitochondrial nucleic acid, nucleic acids from infectious organisms, messenger RNA, micro-RNA, or nucleic acid drug.

In the following description, the terms “polypeptides” is used in its most general sense, and design in particular any kind of molecule, or molecular assembly comprising aminoacids sequences, natural and artificial proteins, polypeptides, fragments of proteins, protein complexes, enzymes, antibodies, glycopeptides, or glycoproteins. and their chemical or biochemical modifications

As used herein, the term “ligand” represents a species, or a function, able to bind reversibly or irreversibly with another species, in particular an analyte. Numerous ligands are known from those skilled in the art. Of particular interest as ligands within the invention are antibodies, metals, histidine tags, hydrophobic moieties, hydrogen-bonding capture moieties, protein A, charged species nucleic acid sequences, polyelectrolytes, phospholipids, chemicals, drugs, nucleic acids, antibodies, fluorescent moieties, luminescent moieties, dyes, nanoparticles, gold nanoparticles, quantum dots, DNA intercalating dyes, aptamers,

As used in the present description, the term “analyte” may represent any compound or material entity one wishes to separate from a sample, to study, to analyse, to store, or to cultivate. Analytes within the inventions may be molecules, ions, atoms, macromolecules, and particularly macromolecules, or analyte colloidal objects. By the term “analyte”, one may designate indifferently one single kind of species, or a multiplicity of kinds of species, present in a sample.

As used in the present description, the term “analyte colloidal objects” may represent a large variety of compounds, including cells, organelles, viruses, cell aggregates, cell islets, embryos, pollen grains, artificial or natural organic particles such as latex particles, dendrimers, vesicles, magnetic particles, nanoparticles, quantum dots, metal microparticles, metal nanoparticles, organometallic micro or nanoparticles, nanotubes, artificial or natural macromolecules, microgels, macromolecular aggregates, proteins or protein aggregates, polynucleotides or polynucleotide aggregates, nucleoproteic aggregates, polysaccharides, or supramolecular assemblies, or combinations of the hereabove compounds. The term “analyte particle” will be used in the description with the same meaning as “analyte colloidal object”.

As used herein, “microfluidic,” “microscopic,” “microscale,” the “micro-” prefix (for example, as in “microchannel”, and the like) may refer to elements or articles having widths or diameters, or at least one of their dimensions, of less than about 1 mm, and less than about 100 microns (micrometers) in some cases. Additionally, “microfluidic,” as used herein, refers to a device, apparatus or system that includes at least one microscale channel.

A “channel”, as used herein, means a feature on or in an article (e.g., a substrate) that at least partially directs the flow of a fluid. In some cases, the channel may be formed, at least in part, by a single component, e.g. an etched substrate or molded unit. The channel may have any cross-sectional shape, for example, circular, oval, triangular, irregular, square or rectangular (having any aspect ratio), or the like, and may be covered or uncovered (i.e., open to the external environment surrounding the channel).

In embodiments where the channel is completely covered, at least one portion of the channel may have a cross-section that is completely enclosed, and/or the entire channel may be completely enclosed along its entire length with the exception of its inlet and outlet.

A channel may have in at least some of its sections an aspect ratio (length to average cross-sectional dimension) of at least 2:1, more typically at least 3:1, 5:1, or 10:1. As used herein, a “cross-sectional dimension”, in reference to a fluidic or microfluidic channel, is measured in a direction generally perpendicular to fluid flow within the channel. In an article or substrate, some or all of the channels may be of a particular size or less, for example, having a largest dimension perpendicular to fluid flow of less than about 5 mm, less than about 2 mm, less than about 1 mm, less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm or less in some cases. However, as will be made more apparent in the following detailed description of the invention, the present invention may also involve a largest dimension perpendicular to fluid flow not current in conventional microfluidic systems, e.g. larger than 1 mm, larger than 5 mm, larger than 1 cm, or even larger than 3 cm, 5 cm or 10 cm.

In one embodiment, the channel is a capillary channel. However, in some cases, larger channels, tubes, etc. may be used to store fluids in bulk and/or deliver a fluid to the channel.

The term “microsystem” as used herein, refers to a device involving deliberate and functional microstructures, prepared by a process involving in one of its step microfabrication of self-assembly.

The term “microfluidic” as used herein to further qualify a microsystem is to be understood, without any restriction thereto, to refer to structures or devices through which fluid(s) are capable of being passed or directed, wherein one or more of the dimensions is less than 500 microns. In some embodiments, microfluidic systems involve microchannels.

The term “microchannel” as used herein is to be interpreted in a broad sense. Thus, it is not intended to be restricted to elongated configurations where the transverse or longitudinal dimension greatly exceeds the diameter or cross-sectional dimension. Rather, such terms are meant to comprise cavities, tunnels or three dimensional structures of any desired shape or configuration. Such a cavity may, for example, comprise a flow-through cell where fluid is to be continually passed or, alternatively, a chamber for holding a specified, discrete amount of fluid for a specified amount of time.

As used herein, the term “microchannel network” refers to one or more microscale channels that are disposed between two substrates, or integrally surrounded by a substrate, and are in fluid communication, or may be put in fluid communication with each other thanks to a microvalve integrated in the substrate.

The term “microchannels array” designates an ensemble of at least two, non connected, microchannels or microchannel networks, microfabricated in the same substrate. A microchannels array may involve microchannels that are in addition involved in microchannels networks, thus leading to an array of microchannel networks.

In the following, except if specified otherwise, the term “microchannel” will be considered as comprising either a single microchannel, a multiplicity of microchannels, a microchannel network or a microchannel array.

The active zone of a microchannel is defined as a zone of a microchannel that carries on at least one of its surfaces at least one direct or indirect capture domain, or capture element suitable for direct or indirect capture of analytes, respectively. In the following, the names “active zone” or “active area” will be used indifferently with the same meaning.

By “capture”, we mean the deliberate immobilization of an analyte in at least one predefined zone of the microfluidic device.

The two terms, “capture domain” or “capture element”, will be used later on to mean a specific portion of the active zone of the device, where or on which analytes may be directly or indirectly captured. This capture may involve, for instance, direct contact between said analyte and said capture element, or contact of said analyte with a surface belonging to a secondary element, hereafter called “capture objet” such as for instance a capture colloidal object, itself immobilized on said capture element. Preferably, then, said capture object is itself immobilized in the active zone onto a capture domain, in such case the capture of analytes is said indirect

A capture colloidal object is, within the invention, a colloidal object that can be immobilized in an active zone within a device according to the invention, and that can itself bind an analyte. Said capture colloidal object may be of various natures, such as a latex bead, a microparticle, a nanoparticle, a microgel, a dendrimer, a vesicle, a liquid droplet. It can also be of various materials, among which mineral, organic or organomineral materials, and more specifically, for instance, polymer latexes, metals, metal oxides, ceramics, silica, glass, organic liquids, hydrogels, and combinations thereof.

For instance, if the active zone is activable with a magnetic field, said capture colloidal object will preferably be a magnetic microparticle or a magnetic nanoparticle, such as proposed by various companies known from those skilled in the art, Dynal, Miltenyi, Estapor, polysciences, Ademtech, and others . . . . For some purposes, however capture objects with particularly suitable properties for the invention, may be synthetized ad hoc, so the invention is by no means restricted to be used with existing categories of micro or nanoparticles.

The size of said capture colloidal objects can vary within the invention, related to the fact that they can be immobilized on capture elements by various means and in various numbers. In one particular embodiment described in part 4/ below, said capture colloidal objects or capture colloidal objects are bound as single chain, in such case they are preferably in the micrometer range, say between 0.5 μm and 100 μm, preferably between 1 μm and 10 μm, and even more preferably between 1 μm and 6 μm. In other embodiments, however, they can be assembled as columns, and can be as small as 100 nm, and in some cases even as small as 50 nm, or even in more rare cases as small as 20 nm.

Preferably, too, said capture colloidal objects or capture elements are capable to bind at least one type of analyte. In some preferred embodiments, they bear on their surface ligands of said analyte

For terseness, except when explicitly stated otherwise, in the following the term “bead” will also designate capture colloidal objects or capture elements according the above definition.

Exemplary embodiments of the invention thus enable to immobilize and to study analytes, which may be particularly suitable for applications regarding analyte colloidal objects, and notably cells.

In order to apply exemplary embodiments of the invention, analytes are for example flown inside a microchannel, a microchannels network, or a microchannels array, that contains at least one active zone.

Active zones in microchannels of the invention may be of any size and shape, for example parallelepipedal. In other embodiments, they may also be curved, and follow for instance a circle or a fraction of a circle. The active zones may take a large variety of thicknesses.

The thickness of the active zones may be in relation with the distance between capture. The term distance, relating e.g. to capture colloidal objects or capture elements, relates to the distance between their centers of mass.

In some preferred embodiments, the thickness of the active zones is comprised between 0.5 times and 10 times the distance between capture elements. In some other preferred embodiments, said thickness is comprised between 5 times and 100 times the size of capture elements.

In preferred embodiments, when analytes of interest are cells, the distance between capture elements is comprised between the average diameter of said cells, and 20 times the average diameter of said cells, preferably between 2 times and 10 times said diameter. For the sorting of human cells, for instance, distances between capture elements centers of mass will be comprised between 30 μm and 100 μm, and preferably between 40 μm and 80 μm, even more preferably between 50 μm and 70 μm.

Sizes and distances above are considered in a general sense.

As used herein, the term “size”, when referring to a particle or analyte, relates to its dimensions in a plane encompassing its center of mass.

For instance, the invention encompasses embodiments in which either the size, or the spacing, or both, of capture elements vary, either regularly, or irregularly, or randomly within one microchannel, or from one microchannel to the other if the embodiment involves several microchannels. Thus, the preferred specifications recited above may concern only a subset of all capture elements in a given embodiment, and the invention may exert its benefits even if some capture elements are out of the range of said specifications. Except when specified otherwise, when reference to the size of a capture element, or to the distance between magnetic domains, is made in the text, and said size or distance vary within the microsystem of the invention, reference is made to the average size or distance.

In exemplary embodiments for the sorting of cells from mammals, the thickness of active zones is comprised between 20 μm and 100 μm, in particular between 40 and 80 μm, for example between 50 and 70 μm.

Preferably, in the invention, at least one of said active zones is sealed, on one of its side, by a layer of a transparent material with a thickness suitable for high resolution microscopy observation. Said layer will be called in the following the “window”. In exemplary embodiments, said window has a thickness smaller than 500 μm, preferably smaller than 200 μm. In a particularly suitable embodiment, said layer is mainly made of glass, and has a thickness equal to the standard thickness of microscope coverslips. In other suitable embodiments, as will be made more clear below, said layer can also be made of, or comprise, a transparent polymer. Such polymer can be an elastomer, such as polydimethysiloxane, or fluorinated polymer such as “Dyneon”. Said polymer may also be a thermoplastic polymer, such as olefin polymer or copolymer, notably cyclic olefin copolymer, polycarbonate, polymethyl methacrylate, polystyrene, polyethylene terephtalate, The polymer cited above are only cited for convenience and exemplary demonstration, and should not be considered as a limitation of the invention. Indeed, numerous transparent polymers are known by those skilled in the art, and can be used within the invention depending of its particular application, alone or in mutual combination or in combination with another transparent material such as glass or silica.

The thickness of said active zone may be comprised on at least part of its surface between 20 μm and 100 μm, between 40 and 80 μm, or between 50 and 70 μm.

According to other exemplary embodiments of the invention, the combined thickness of said window and said active zone, in regard of said window, is smaller than 300 μm, and preferably smaller than 250 μm.

Such a thickness may be particularly suitable for high resolution microscopic observation.

At least one portion of the active zone may be bounded on two of its sides facing each other by transparent material.

In one embodiment, said active zone comprises on at least one of its surfaces a capture element arranged to perform direct or indirect capture of analytes, and preferably an array of such capture elements

By “direct capture of analytes”, we mean that analytes are immobilized, or bound, in direct or close contact of said capture elements.

By “indirect capture of analytes”, we mean that the active zone is able to immobilize secondary elements, said secondary elements being able to capture or bind said analytes at their surface. Example of such secondary elements may be microparticles or nanoparticles, or more generally capture colloidal objects or capture objects.

In embodiments where capture of analytes is direct, capture elements are for example patches of ligands to said analytes. As used herein, ligands represent a species, or a function, able to bind reversibly or irreversibly with another species, in particular an analyte. Numerous ligands are known from those skilled in the art. Of particular interest as ligands within the invention are antibodies, e.g. antibodies directed towards surface antigens of the COI However, numerous other ligands may be used, such as metals, histidine tags, hydrophobic moieties, hydrogen-bonding capture moieties, protein A, and the like. Other types of ligands that may be used are ligands based on nucleic acids, and able to bind specifically to some nucleotidic sequences.

Ligands such as e.g. polyelectrolytes, or phospholipids, may also exert their capture thanks to electrostatic interactions.

Ligands may also represent chemicals, drugs, nucleic acids, combinations of nucleic acids and enzymes, such as mixtures used for DNA amplification, antibodies, fluorescent moieties, luminescent moieties, dyes, nanoparticles, gold nanoparticles, quantum dots, DNA intercalating dyes, aptamers, or any types of species putatively able to affect the metabolism of cells, or the properties of colloidal objects according to the invention, in particular their optical properties.

Such ligands may be attached to a surface of a microchannel or of a colloidal object.

Such ligands may be configured to perform a reversible or irreversible capture of the analytes. By “irreversible capture”, we mean the capture of a species, for instance an analyte or a colloidal object, which cannot be released without destroying or altering in an important manner, the integrity of said analyte or object. A typical example of irreversible capture is bonding by a chemical covalent link. In some cases, however irreversible capture may be obtained without covalent bonding, for instance when proteins are denatured on a surface, or latexes are irreversibly attached to a surface by drying or heating.

By “reversible capture”, oppositely, we mean a capture that may be released without significantly modifying the bound species. Reversible capture may be due to physical means, such as for instance capture of two magnetic particles by the activation of a magnetic field, or capture by hydrophobic interactions, or electrostatic or dielectrostatic force. Reversible capture may also be due to chemical means, such as hydrogen bonding, or reversible chemical reaction. Finally, reversible capture may be due to biochemical interactions, such as hybridization of nucleic acid strands, antigen-antibody interaction, aptamer-protein interactions.

In other exemplary embodiments, capture is physically or chemically activable.

A capture is called “physically activable”, if it may be triggered by a modification of a physical parameter, such as temperature, magnetic field, electric field, light, or flow field.

A capture is called “chemically activable”, if it may be triggered by change in a chemical state, such as pH, redox potential, or ionic strength, or presence of some specific ions or molecules, e.g. tensioactive agents, or enzymes.

In embodiments wherein capture is physically activable, capture elements may, for instance, be magnetic domains or conductive domains.

Magnetic domain designates a volume or a surface, with a delimited perimeter, constituted of magnetic material, or comprising magnetic material, such as superparamagnetic material, ferromagnetic, ferrimagnetic, or antiferromagnetic material. Any kind of magnetic materials, such as metals, metal oxides, ferrofluids, may be used to prepare magnetic domains within the invention. In some preferred embodiments of the invention, such magnetic domains are used as an array organized on a surface of a microchannel.

In one exemplary embodiment, the capture elements are magnetic and the device comprises means to apply an external magnetic field to the active zone, in such a way that the capture elements are activable, and preferably reversibly and physically activable.

Said means may involve coils, permanent magnets, and optionally cores made of soft magnetic material. If said means involve permanent magnets, they may comprise a mobile magnetic shunt, in order to allow or prevent the flow of magnetic field lines across said active zone, by mechanical relative displacement of said shunt and said magnetic material.

In some embodiments said magnetic field is essentially uniform in a given active zone.

In some embodiments, said magnetic field is along a direction transverse to the general flow direction of sample fluid in the microchannel, and to the surface of the window. Said field is for example perpendicular to said flow direction and to said window.

The capture elements are for example activated thanks to their higher magnetic permittivity than that of the surrounding medium. This way, as shown in more detail in the examples, then may focalize magnetic field lines, and create local magnetic field gradients able to capture magnetic objects, such as magnetic particles.

Preferably, said external magnetic field is comprised between 5 mTesla and 50 mTesla, and preferably between 15 mTesla and 40 mTesla.

According to other exemplary embodiments, capture elements are electrically conducting and the device comprises means to induce in the active zone a DC or AC field, or A DC or AC current, such means enabling the capture elements to be made reversibly activable.

The capture elements are for example directly connected to an electric current or field generator.

An electric field or electric current may be induced in said active zone by using activating electrodes located outside of said active zone. In this later embodiment, capture elements may become active, because of their higher conductivity as compared to the environment, focalize electric field lines, and create electric field gradients that attract charged compounds if the field comprises an DC component, or that attract polarizable material if the field comprises an AC component.

This later effect, called dielectrophoresis, is well known from those skilled in the art, who may tune the field properties, such as amplitude and frequency, in order to attract or repel specified analytes or objects, using their complex permittivity spectrum as described e.g. in Braschler et al., Lab Chip 2008, 280-6).

In some embodiments, the capture elements may not act as obstacles, so that they do not significantly hinder the passage of fluids and analytes in the channel, such a property being useful since it enables automated operation of devices according to the invention.

In some embodiments, said capture elements are not functionalized, i.e. they do not bear ligands. In some other preferred embodiments, however, they may within the invention carry ligands.

The capture elements may be of any shape.

Said capture elements may be arranged in a regular, symmetrical array, although some applications may require the use of asymmetrical, or even irregular arrays.

The capture elements within the invention may be of any nanometric or micrometric size. They may for instance be of a size comprised between 10 nm and 50 nm, or between 50 nm and 200 nm, or between 200 nm and 500 nm, or between 500 nm and 1 μm, or between 1 μm and 2 μm, or between 2 μm and 5 μm, or between 5 μm and 10 μm, or between 10 μm and 20 μm, or between 20 μm and 50 μm, or between 50 μm and 100 μm, or between 100 μm and 1 mm. For the capture of cells, said capture elements may have a size comprised between 1 μm and 20 μm, and preferably between 2 μm and 10 μm.

Also the spacing between said capture elements may vary depending on the analytes to be separated, and on the species present in the sample besides said analytes. Spacing between the center of mass of capture elements may be comprised between one time and a hundred times the size of the capture elements, for example between 2 times and 50 times that size, in particular between 5 times and 20 times that size.

Capture elements may be involved in the invention as microfabricated or micropatterned layers on a surface, or deposited on a surface, using for instance microcontact stamping, or as microparticles or nanoparticles irreversibly attached to said surface.

Active zones in microchannels of the invention may be of any size and shape, being for example essentially parallelepipedal. Other interesting layouts of active zones may be a circular strip, or a portion of a circular strip, as will be exemplified in FIG. 5. They may take a large variety of thicknesses.

At least some of capture elements may be organized as a layer at the internal surface of the active zone's window. Said layer is for example at the bottom of the microchannel. Depending of applications, however, microchannels according to the invention may have different dispositions, with a window below or above the microchannel (as reference to earth gravity).

In some embodiments, the footprint of the complete microfluidic circuit may preferably be smaller than 12 cm², being for example smaller than 10 cm².

In other embodiments, particularly suitable for mass production, the footprint of the complete microfluidic circuit may have the shape and size of a CD, or of a mini-CD.

By “footprint of a microfluidic device”, or by “footprint of an active zone”, is designated the area, measured in the plane (or in the surface, if said microfluidic device is not planar), in which said active zone, or the microchannels of said microfluidic device, lie.

While combining a minimal thickness of said microchannel in said active zone, a minimal footprint of the complete microfluidic circuit, and a maximal flow rate, the invention makes it easier to capture and study analytes in the active zone.

Where appropriate, the footprint of the total active area is smaller than 1 cm², comprised between 1 and 2 cm², or between 2 and 5 cm², and in some cases comprised between 5 and 10 cm². Reducing the footprint of the microfluidic device and the footprint of the active area may be advantageous since it decreases the area that has to be scanned by optical tools for the automated screening of large samples.

As a convenience feature for keeping its footprint minimal, and described in FIG. 6 and related text, the microfluidic device may comprise:
a first layer of microchannels comprising at least a first microchannel in direct contact with the window and,
a second layer of microchannels, essentially parallel to said first layer,
   wherein the projection of at least one microchannel in said second layer along a direction perpendicular to the plane in which said first layer is located, crosses the projection of at least one of the microchannels comprised in said first microchannels, without fluidic connection between said microchannel in said second layer and said microchannel in said first layer at the position of crossing.

The microfluidic device may comprise at least one inlet and one outlet, in a configuration suitable to induce flow in said microchannel in a direction essentially transverse to the largest dimension of said microchannel or of said active zone.

Other exemplary embodiments of the invention provide a device, being optionally any microfluidic device as defined above, for the sorting of analytes, comprising:
at least one microfluidic channel comprising at least one active zone carrying on at least a portion of one of its surfaces an array of capture domains, and
at least one inlet and one outlet, in a configuration suitable to induce flow in said microchannel in a direction essentially transverse to the largest dimension of said microchannel or of said active zone.

Such a microfluidic device may be part of a unit further comprising at least one any above-defined microfluidic device.

Said at least one inlet and at least one outlet may be composed of several subsidiaries from a primary inlet and a primary outlet, respectively, arranged in order to direct equivalent quantities of fluid to equivalent cross-sectional areas of the active zone, or of the active zones, which may enable the flow of sample fluid across the active zones not to have large variations, thereby improving the uniformity and efficiency of capture. In a variant, the device may comprise several microfluidic channels working in parallel. By splitting the volume in which analytes are sorted, the uniformity and efficiency of capture may be improved.

Some examples of layouts of microchannels suitable for implementing the invention are provided in FIG. 5.

Within the invention, it is believed that capture of analytes is more uniform and more efficient if analytes interact with all binding elements or all capture colloidal objects with approximately the same speed. Thus, preferably the velocity of flow, measured in the midplane of said active zone with regards to the thickness of said active zone, along a line essentially perpendicular to flow direction, should not vary by more than 30%, and preferably should not vary by more than 20%, around its average value, in at least 90% of the length of said line

It will be apparent to those skilled in the art, and made more apparent in the examples below, that said multiplicity of microchannels, is a conveniency of fabrication, but that they are operationally equivalent to a single equivalent microchannel with a section equivalent to the combined section of said microchannels, section being defined here as the cross area perpendicular to the general flow direction.

Other exemplary embodiments of the invention provide a microfluidic device for the sorting of cells, optionally any microfluidic device as defined above, comprising:
a first layer of microchannels comprising at least a first microchannel in direct contact with a window made of transparent material with a thickness smaller than 500 μm, and,
a second layer of microchannels, essentially parallel to said first layer, wherein the projection of at least one microchannel in said second layer along a direction perpendicular to the plane in which said first layer is located, crosses the projection of at least one of the microchannels comprised in said first microchannels, without fluidic connection between said microchannel in said second layer and said microchannel in said first layer at the position of crossing.

Such a microfluidic device may be part of a unit further comprising at least one any above-defined microfluidic device.

Other exemplary embodiments of the invention provide a device, optionally any microfluidic device as defined above, for the sorting of analytes, comprising a series of at least one microchannel, configured to receive in parallel a flow of a liquid containing said analytes, wherein said at least one microchannel carry on at least a portion of one of its surface an array of capture elements, and

wherein the combined width (measured perpendicular to flow direction) of said at least one microchannel is larger than their effective length (measured parallel to flow direction).

Such a microfluidic device may be part of a unit further comprising at least one any above-defined microfluidic device.

The effective length is aligned along the general direction of flow and the width is perpendicular to said general direction of flow.

The combined width of said at least one microchannel may be larger than twice, for example 5 times, in particular 10 times, their effective length. In other embodiments, though the ratio of this combined width to said effective length can exceed such values, and be e.g. larger than 10, 20 or even 50 or 100.

The device may comprise several active zones of a similar length.

In a variant, the active zones may be of different lengths, the effective length being in such a ease measured as the average length of said microchannels, the average being considered as referenced to the cross section of the active zone of said microchannel.

The combined width of said at least one active zone may be larger than their effective length, for example larger than twice their effective length, in particular 5 times or 10 times their effective length, the effective length and width being defined as above.

The invention may allow to flow large volumes of samples, in active areas with a small footprint.

Microchannels in devices of the invention may be made of any material, and made with any microfabrication process. Numerous methods for microfabrication, and numerous materials usable for the fabrication of microfluidic networks, are known from those skilled in the art, and may be used within the invention (see e.g. Zadouk, R., Park BY, Madou, M J, Methods Mol. Biol. 321, 5, (2006). As an exemplary but non exclusive list of usable materials, microfluidic systems of the invention may be constructed polydimethyl siloxane, siloxane elastomers, other elastomers, thermoplastics such as polystyrene, polymethyl methacrylate, cyclic olefin copolymer, polyamide, polyimide, thermosetting or photopolymerizable resins, polymerizable or photopolymerizable gels, such as acrylate compounds, PEG-acrylate compounds, ceramics, silicon, glass, fused silica, or combinations of these materials.

In a preferred family of embodiments, said materials may be transparent to light, or partly transparent to light, the transparent part of said embodiment being called the “window”. The window is for example made of glass, or transparent polymer.

Other parts of the microfluidic device, located opposite of the window with regards to the active zone, may also be made of transparent material in some preferred embodiments.

The substrates carrying the microfabricated networks within the invention may have any shape. They may be planar, or comprised in a developable substrate, such as a polymer film or sheet.

Optionally, they may be deformable, and may comprise microfabricated valves, pumps, membranes, filters, microstructures, integrated optics, electrodes, detection units, surface treatments, and all kinds of microfluidic components and technologies, as recited e.g. in Micro Total Analysis Systems 2005, K. F. Jensen, J. Han, D. J. Harrison, J. Voldman Eds, TRF press, San Diego, Calif., USA

Microchannels of exemplary embodiments of the invention may involve a variable thickness, or a discrete set of more than one thickness. In particular embodiments, they involve at least one first type of microchannels, which encompass the active zone(s) with a first thickness, and a series of second microchannels, or “feeding” microchannels arranged to distribute fluids to at least one inlet of said first microchannel, and to collect fluids from at least one outlet of said first microchannel, said second microchannels having a thickness larger than that of said first microchannel.

Other exemplary embodiments of the invention provide an instrument for the capture and study of colloidal object, and particularly cells, said instrument comprising:
a microfluidic device as defined above and,
at least one microscope objective with an optical axis perpendicular to the window of the microfluidic device,

wherein the instrument is configured so that colloidal objects are flown into at least one microchannel of the device, and

wherein the objective is configured so that images of the content of the active zone of the microfluidic device may be observed or recorded across said window.

The microscope objective may have a magnification larger than 18×, in particular larger than 35×, for example larger than 59×, and in some embodiments as high as 100×.

The microscope objective may have a numerical aperture of at least 0.2, for example as high as 0.4, for example as high as 0.6, as high as 0.8, as high as 1.0, as high as 1.3, or even as high as 1.4.

The use of high numerical aperture, high magnification objectives, could not be implemented in cell sorting devices of prior art, and are thus a distinctive advantage of exemplary embodiments of the invention.

The instrument may also comprise one of an optical 3-dimensional imaging device, an optical sectioning imaging device, a holographic imaging device, a spinning disk imaging device and a confocal microscope imaging device.

“3-dimensional imaging” designates reconstituting 3 dimensional images of the observed field. 3-dimensional imaging comprises, as a non exhaustive list, confocal imaging, two-photon scanning imaging, spinning disk imaging devices, deconvolution microscopy, or imaging methods based on structured illumination.

“Optical sectioning imaging” designates a mode of imaging of a volume, wherein said volume is represented as a stack of images, each of said images in said stack corresponding to a given layer in said volume.

The invention may thus allow the implementation of 3-dimensional imaging of the captured analytes, or imaging of said analytes using optical sectioning.

Notably, too, thanks to its flexibility and power for optical imaging, the invention can advantageously be used synergistically with a variety of spectroscopic or spectroscopic imaging methods and tools, in order to characterize the captured analytes with these tools. These tools can advantageously be InfraRed (IR) spectroscopy, Fourier Transform Infra Red (FTIR) spectroscopy, IR and FTIR imaging spectroscopy, Scanning Force Microscopy, Plasmon Resonance, Plasmon Resonance Imaging, spectroscopic imaging and hyperspectral imaging spectroscopy, Raman spectroscopy, Raman Imaging Spectroscopy, Surface Enhanced Raman Spectroscopy (SERS), Fluorescence Resonance Energy Transfer (FRET), Luminescence energy transfer methods such as BRET, and the like.

The invention is also advantageously combined with time resolved versions of the above imaging or spectroscopy methods, notably time resolved luminescence and fluorescence, or time resolved imaging fluorescence or luminescence imaging

In some preferred embodiments, the analysis or imaging operations recited above are performed directly in the active zone. In other embodiments, however, they can be performed in a different observation zone, after release and transport of said analytes from said active zone. This is made particularly convenient in the invention thanks to the reversibly activable nature of the capture elements,

Other exemplary embodiments of the invention provide a system for the capture and study of analytes, and particularly analyte colloidal objects or cells, wherein said analytes are flown into at least one microchannel comprising at least one active zone comprising at least one activable or reversible capture element.

Preferably, the device may be arranged so that the active zone may be moved in the field of observation of the imaging device.

Within microfluidic devices of the invention, the analytes are not attached to a surface of said microfluidic device, as in previous optical cytometry systems, but to particles, so that they may better keep their 3-dimensional shape. This is particularly advantageous when analytes are cells.

The invention may enable to investigate how biomarkers are arranged within this 3-dimensional shape.

“Biomarker” is used here to designate any type of information that may be gained on the biological state or on the condition of an organism. For instance, and as a non-exhaustive list, classical biomarkers may be: the presence of a protein; the expression of a protein in a given tissue, body fluid, cell, or cell compartment above or below a given threshold; the expression of a gene or a combination of genes; a mutation; a phenotype; a morphological characteristic; the presence or absence of some types of cells, or of some types of proteins, or of some type of ions or molecules, in a body fluid, an organ, a cell, a cell compartment; the proliferative power of a cell, or a cell assembly; the response of a cell, a tissue, an organism, to a chemical or physical stimulus. A biomarker may for example be identified using a biomolecule bearing a label

Within the invention, a label may be any kind of molecule, moiety or particle, that may be identified specifically with a physical or chemical means. As a non restrictive exemplary list, labels in the invention may involve fluorescent groups, luminescent groups, chemiluminescent groups, electroluminescent groups, quantum dots, metal nanoparticles and notably gold or silver nanoparticles or quantum dots, coloured molecules, electroactive groups, molecules able to be recognized by an antibody or a peptide sequence, such as biotin, digoxigenin, Nickel, histidine tags. Labels also involve enzymes able to turn a substrate into a detectable products, like in ELISA based assays. The detection of said substrate may be colorimetric, by UV or visible absorption, fluorescent, electrochemical, or involve any kind of optical or electronic imaging or detection method.

Several cells may be arranged in the invention so that their images in a conventional 2D imaging, as performed in prior art, overlap. In such case, without 3 dimensional imaging or optical sectioning, it would be difficult to know to which cells belongs a given biomarker seen on the overlapping image. The possibility of performing on the analytes 3D imaging, is thus a definite advantage of the invention.

Finally, in some embodiments, it may be advantageous to reconstitute 2 dimensional images of cells, from the 3 dimensional images or optical sectioning stacks, described above. Very surprisingly, this provides better characterization than direct 2D images. This is particularly advantageous if, during the reconstitution of a 2D image pertaining to a given cell, only a subvolume of the 3D image, or only a subset of the optical sectioning stack, is used. This way, one may get on a 2D image only information pertaining to a given cell, and avoid unwanted signal (for instance fluorescence) from the microchannel wall, from other cells, or from microparticles or nanoparticles.

Other exemplary embodiments of the invention provide a method for storing, screening, study or culture of analytes, and notably cells, wherein said analytes are flown across a microfluidic device or an instrument as defined above.

Other exemplary embodiments of the invention provide a method for optical screening of analytes, and notably cells, contained in a sample, comprising:

a/ Flowing said sample in at least one microchannel, optionally of any microfluidic device as defined above, carrying on at least one of its surfaces capture elements

b/ performing an optical imaging of the captured analytes in said microchannel, resulting in a multiplicity of images corresponding to different cross sections of said analytes.

Optionally, step b may be followed by a step c, wherein a subset of said images are selected and combined in order to reconstitute a 2 dimensional image. Said imaging is for example multicolour, i.e. said images involve at least two, preferably 3, and even preferably 4 or 5, different “optical channels” corresponding to either different excitation wavelengths, or different emission wavelengths, or different combinations of excitation and emission wavelengths.

Optionally, too, the level of light emission from different captured analytes in these different optical channels, are compared and or/quantified, in order to evaluate for the presence or concentration or level or expression or distribution of biomarkers of interest.

Optionally, too, step b may be replaced by a more conventional step of 2D imaging, in which the invention still provides advantages with regards to state of the art methods, notably thanks to its ability of performing imaging with objectives presenting a high magnification or a high numerical aperture or a combination of both, thanks to its small footpring, high selectivity, limited damage to cells, and other advantages to be described later on in this application.

The analytes may be flown across in microfluidic device according to the invention, with an active zone presenting a footprint smaller than 8 cm², for example smaller than 5 cm², and in some cases smaller than 2 cm².

The analytes may be flown at a flow rate of at least 20 μL/hour, for example 50 μL/hour, in particular 100 μL/hour, 200 μL/hour, 500 μL/hour, 1 mL/hour, 2 mL/hour, and up to more than 5 mL/hour.

Other exemplary embodiments of the invention provide a method for the sorting, study, storage or culture of analytes, said method comprising:

a/ providing a microfluidic device, optionally any microfluidic device as defined above, comprising at least one microchannel comprising at least one active area comprising at least one activable capture domains, said microfluidic device comprising additionally first means to activate said activable capture domains and second means to controllably flow fluids in said microchannel,

b/ flowing in said at least one active area capture colloidal objects able to assemble onto said capture domains upon activation of said first means, and bearing ligands for said analytes,

c/ activating said first means, and

d/ flowing in said at least one active area a fluid sample containing said analytes.

The method may comprise in addition a rinsing step e, performed between steps c and d above, in which said active area is rinsed with a fluid containing no capture colloidal objects able to assemble onto said capture elements, and no analytes.

The method may further contain additional steps f, following step d, in which reagents are flown into said active area whereas said first means are kept activated.

The method may further contain the following step:
moving said microfluidic device from a first instrument in which the capture of analytes is performed to a second instrument in which the analysis or imaging of analytes is performed.

Said reagents may be reagents for revealing biomarkers, such as antibodies or nucleic acid probes. These reagents, antibodies or nucleic acid probes may be labelled with enzymes, or with luminescent, fluorescent, electrochemical, diffusive, or radioactive labels, or with quantum dots, or with gold nanoparticles, or with color dyes, or more generally any ligand that can be detected by physical, chemical, biological or biochemical method. If said reagents involve enzymes, an additional step involving flowing in said active area substrates for this enzyme, is generally preferred.

Said reagents may be ligands to said analytes bound to at least one label selected among colored dyes, fluorescent groups, luminescent groups, chemiluminescent groups, electroluminescent groups, quantum dots, metal nanoparticles and notably gold or silver nanoparticles or quantum dots, coloured molecules, electroactive groups, molecules able to be recognized by an antibody or a peptide sequence, such as biotin, digoxigenin, Nickel, histidine tags, or enzymes, or substrates for enzymes.

Optionally, the method further comprises a supplementary steps g, in which reagents able to fix or permeabilize cells are flown in said active area, in order to perform subsequently, as an exemplary and non-limitative list, fluorescence in situ hybridization to check for genetic integrity or mutations in captured cells, RNA or DNA amplification, or search of intra-cytoplasmic biomarkers.

Many ways to screen captured biological analytes, and particularly cells, for biomarkers are known by those skilled in the art, especially cellular biologists and pathologists, and may be implemented within the invention. The invention, may allow the application to captured rare cells, of sophisticated cell screening protocols that are currently applicable only to cells in culture.

Some non-restrictive examples of such screening methods applicable within the invention, will be described in the examples below

Optionally, exemplary embodiments may also involve additional step h of flowing, after step d, into the active area, a drug, or more generally a chemical, in order to screen the response of captured analytes, and notably captured cells, to said drug or chemical.

Optionally, exemplary embodiment may involve a step i of flowing into said active area a mounting agent, or a hardenable material, in order to immobilize the captured analytes and capture colloidal objects, and keep them immobile for observation even after deactivation of the means for activating the activable capture domains. This allows for instance to move said microfluidic device from a first instrument in which the capture of analytes is performed, to a second instrument in which the analysis or imaging of analytes is performed, as made more clear for instance in part 12/ below. Numerous mounting agents and hardenable materials are known from those skilled in the art, and may be used within the invention, depending on the characteristic of the analytes. As a non restrictive list, such hardenable materials may be a solution of polyvinyl alcohol, PVA, or agarose, or acrylamide, or PEG-acrylate.

As used herein, a material is said “hardenable”, if it is capable of undergoing a transition to a solid state, a gel state, a viscoelastic state, and generally speaking a state in which it is able to keep its shape after application of a stimulus, as opposed to the behavior of a liquid.

This may be achieved by using as hardenable material a polymerizable material, or a crosslinkable material. More preferable, this polymerization or crosslinking may be triggered in the microchannel network, e.g. by photoactivation, e.g. if said polymerizable material contains a photoactivator, or by thermal activation, e.g. by bringing at least a part of said microchannels network to a high temperature, if the material hardens upon heating, or opposedly a low temperature, if the material hardens upon cooling. As a second embodiment, this may be achieved by using as hardenable material a material that may change its viscosity or elastic modulus by temperature.

As an example, the hardenable material could be a melted material, that may recover a glassy, crystalline or semicrystalline state, by a decrease in temperature. The hardenable material could also be a material that may transit to a gel state by a decrease in temperature, such as e.g. a water suspension of agarose. Oppositely the hardenable material may comprise a material able to gelify by an increase in temperature, such as poly-N-Isopropyl Acrylamide (PNIPAM).

Various additional ways of hardening material, usable for the invention, are recited e.g. in U.S. Pat. No. 6,558,665 to Cohen, or in the “Polymer Handbook”, J. Brandrup et al. eds, Wiley, incorporated herein by reference.

Hardening of said material may also be obtained by a combination of above effects, first hardening the material by a fast thermal effect, and then making the hardening irreversible by a chemical effect, such as crosslinking or polymerizing. The hardenable material may be selected, depending on the desired application, to be after the hardening step, permeable or impermeable to specific species. The hardenable material may also be hardened by diffusion in said material of a reagent contained in a second fluid by which said first fluid is partly or fully surrounded. In a non-restrictive example, said first fluid may contain sodium alginate, and second fluid may contain oleic acid and calcium chloride. Also, materials known from those skilled in the art of cytology and cytometry under the name of “mounting agents”, may be used as hardenable material within the invention

The above optional steps e to i may be performed in different orders within the invention, including intercalation between some of steps a to d, depending on the analyte under study, and on the properties under study.

As indicated above and shown in some exemplary embodiments below, one of the advantages of the invention, is to allow the application of high resolution imaging tools.

In addition to steps a to d, and optionally to any combination of steps e to i above, the invention may further include at least one of the following steps:
performing high resolution images of captured analytes in the at least one active zone,
performing the characterisation of said analytes using the above-mentioned instrument,
applying image-sharpening algorithms,
applying denoising algorithms and,
applying wavelet analysis.
Said steps may involve the use of a microscope objective with a magnification larger than 35, or larger than 59.

Said images may have a resolution better than 2 μm, for example better than 1 μm, in particular better than 500 nm.

Said images may be 3 dimensional images or an optically sectioned stack of images.

Said images may be collected in an automated way, and stored in an image library.

The whole combined area of the active zone may be imaged in an automated way involving translation of the microfluidic device with regards to a microscope objective.

3 dimensional images are for example obtained at high speed using spinning disk microscopy. Several spinning disk systems known by those skilled in the art, and commercialized by microscope companies such as NIKON®, OLYMPUS®, or ZEISS®, may be used for this purpose. The invention is for example to be used in combination with spinning disk systems, as distributed e.g. by the companies YOKOGAWA®, AUROX®, or others, with which it may be particularly advantageous in terms of cost and compactness.

Thanks to high imaging resolution, exemplary embodiments of the invention are in particular associated with an additional image analysis step, involving image sharpening, denoising, wavelet analysis, and other high performance image tools currently not applicable to sorted analytes, and notably rare cells.

The method may further comprise multicolour labelling and observation of captured analytes,

Said labelling is for example fluorescent, and involves either fluorescent dyes, or quantum dots. Dyes usable within the invention are e.g. Alexa-Fluor, Sybr dyes, cyanine dyes, hoecht dyes.

Staining protocols conventionally used by pathologists, such as, for instance, May-Grunwald-Giesma, may also be used

Other exemplary embodiments of the invention provide a method for diagnosis or prognosis, wherein a sample from a patient is submitted to any above-defined method.

The diagnosis or prognosis may relate to cancer, prenatal diagnosis, genetic diseases or cardiovascular diseases.

The invention allows for example to perform the in situ analysis, of the transcriptome or genome, of captured cells. The analytes may comprise at least one of cancer cells, circulating tumour cells, disseminated tumour cells, circulating foetal cells, circulating endothelial cells, or circulating infectious cells.

For clinical applications, analytes may be initially contained in a sample selected among blood, fine needle aspirates, biopsies, bone marrow, cerebrospinal fluid, urine, saliva, lymp.

In other embodiments, the invention may perform the search and analysis of contaminants or biohazard organisms for environmental applications, e.g. in surface of ground water, industrial liquids, or liquids issued from water treatment systems, or liquids issued from environmental biocollectors.

The method may further comprise performing immunophenotyping of at least one of captured analytes within said active zone.

The method may further comprise analysing nucleic acid sequences in at least one of captured analytes.

Said nucleic acid sequence belongs for example to genomic DNA, messenger RNA, microRNA, ribosomal nucleic acid, mitochondrial nucleic acid, nucleic acid from an infectious organism, or nucleic acid drug.

The method may further comprise analysing polypeptides in at least one of captured analytes.

Said polypeptide or said nucleic acid may belong to a potentially infectious organism.

Other exemplary embodiments of the invention provide a method for screening drugs, chemicals or compounds for their toxicity, efficiency or biological effect, comprising:

a/ flowing a sample containing cells in a microfluidic device, being optionally any microfluidic device as defined above,
b/ flowing in said microfluidic device a solution containing at least said drug, chemical or compound and,
c/ observing or measuring the effect of said drug, chemical or compound on said cells.

Other exemplary embodiments of the invention provide a method for cancer diagnosis or prognosis comprising:

a/ flowing in a microfluidic device as defined above a sample from a patient,
b/ flowing into said microfluidic device a solution suitable for sustaining life of at least one of captured cells and,
c/ assessing the proliferative power of said at least captured cell.

Other exemplary embodiments of the invention provide a method for cancer diagnosis or prognosis comprising:

a/ flowing in a microfluidic device as defined above a sample from a patient

b/ flowing into said microfluidic device at least one solution containing at least one labelling agent allowing to specifically recognize cancer cells or a subpopulation of cancer cells,

c/ quantifying in said microfluidic device the number of cells labelled by said at least one labelling agent.

Optionally the above method may comprise the step of quantifying not only the number of cells labelled, but also the distribution of labelling intensities of said cells.

Other exemplary embodiments of the invention provide a method for cancer diagnosis or prognosis, or a method for screening for the efficiency of a drug or drug candidate, comprising:

a/ flowing in a microfluidic device as defined above a sample from a patient

b/ flowing into said microfluidic device a solution containing a cancer treating agent and,

c/ assessing the effect of said cancer treating agent on said at least captured cell.

Other exemplary embodiments of the invention provide a method for cancer diagnosis or prognosis comprising:
a/ flowing in a microfluidic device, being optionally any microfluidic device as defined above, a sample from a patient
b/ flowing into said microfluidic device a solution suitable for sustaining life of at least one of captured cells
c/ cultivating said at least one of captured cells.

Other exemplary embodiments of the invention provide a method for cultivating, sorting, differentiating or studying stem cells, comprising flowing stem cells into any microfluidic device as defined above.

For all the above applications and others, the invention may be used to study the analytes in situ in the active zone. in some other embodiments, however, the invention may also be used to capture analytes on capture elements or capture objects, and then to release said analytes from said capture elements or objects, and to collect said analytes for further analysis in a second analysis zone, different from the active zone. Said analysis zone may be comprised in the same microfluidic chip as the active zone, or in a different microfluidic device, or even in a different, non microfluidic device. However, having the analysis zone in a microfluidic chip, and especially in the same chip as the capture zone, brings in significant advantages, since it helps, in particular, to take full benefit of the strong reduction of footprint and volume brought in by the invention as compared to prior art, while minimizing risks of contamination and dead volumes.

Other exemplary embodiments of the invention provide a microfluidic device, optionally as defined above, wherein flow of liquid in at least one channel, is controlled at least in part by a valve traversed by said liquid, and

wherein the opening and closing of said valve is progressive, and completed during a first time at least equal to a second time, defined as the time taken by a fluid element to cross the mobile part of said valve, in particular equal to at least twice, in particular equal to five times this second time.

Other exemplary embodiments of the invention provide a microfluidic device

wherein flow of liquid in at least one channel, is controlled at least in part by a valve traversed by said liquid, and
wherein the opening and closing of said valve is progressive, and completed during a time at least 1/10 s, in particular at least ⅕ s, in particular at least half a second, and in some cases at least one second.

Such a microfluidic device may be part of a unit further comprising at least one any above-defined microfluidic device.

Other exemplary embodiments of the invention provide a microfluidic valve of the type in which a tube or a microchannel is pinched by an actuator, such a valve being optionally part of any microfluidic device as defined above

wherein the velocity of this actuator is dynamically controlled by an electronic circuit or a computer, and
wherein the motion of said actuator may be continuous over at least 1/10s, in particular at least ⅕ s, in particular at least half a second, and in some cases at least one second.

Such a microfluidic device may be part of a unit further comprising at least one any above-defined microfluidic device.

The actuator may be made of any material, shape or size, being adapted to the size, shape and material of the microchannel or tubing to be controlled. The actuator has for example the shape of a cylindrical piston, blade, sphere, ellipsoidal element, and may be in metal, ceramic plastic or elastomer.

Other exemplary embodiments of the invention provide an array of valves as defined above, which may enable to automate flow in complex microfluidic networks.

Other exemplary embodiments of the invention provide a microfluidic system comprising an array of valves as defined above.

Other exemplary embodiments of the invention provide a method for the screening of cells, comprising, in addition to steps a to d above, and optionally to some of steps e to i, an additional step j,

wherein at least one type of nucleic acids contained in at least one cell captured in at least one active area, is hybridized with a probe.

Depending on the application, said nucleic acid within said cell may be nuclear DNA to check for point mutation, genetic rearrangements, gene deletion, gene duplication, or chromosomal anomalies, ribosomal DNA, messenger RNA, to check for the overexpression or underexpression of specific genes.

The invention is particularly interesting for the screening in captured cells of microRNA (miRNA) or interfering RNA or silencing RNA, which are difficult or impossible to screen with methods of the art. Some miRNA have been identified as involved in cancer. This is for instance the case for has-mir-155 and has-let-7a-2 involved in lung cancer. Also, for instance overexpression of has-mir-155 or underexpression of has-let-7a-2, is recognized as marker of aggressiveness, and require specific treatments. Of course, one is still at the beginning of research in this field, and these are only non restrictive example of potential applications of the invention for cancer prognosis. The invention will be advantageous for any type of screening regarding diagnosis or prognosis based on biomarkers based on small nucleic acids and specially miRNA, known and yet to be discovered.

Any kind of nucleic acid probes may be used within the invention, in order to detect or quantify nucleic acids of interest in the captured analytes. For instance, said probes may be any kind of natural or artificial nucleic acid or nucleic acid analog, able to hybridize with specific nucleic acid sequence, or a protein recognizing specific at least one nucleic acid sequence

Said probe may bear a label, for instance a fluorescent label, or a luminescent label, or an electrochemical label, or a chemiluminescent label, or an electrochemiluminescent label.

In some exemplary embodiment, said probe bears a ligand, that may itself aggregate a multiplicity of other ligands bearing labels, or able to modify a substrate into detectable products, in order to yield a signal amplification cascade.

In some other exemplary embodiment, the method comprises an additional step k, which may be combined with any of steps e to j above, and notably be performed before step j, said step k involving nucleic acid amplification. Said amplification may be performed in situ in said active area, by any method known in the art, such as PCR, RT-PCR, NASBA, Rolling Circle amplification, LAMP and the like. Notably, a protocol for performing nucleic acid analysis by Rolling Circle amplification on single cells was recited in (Jarvius, et al, Nature Meth, 2006), or in A. Tachihara, et al., Proceedings uTAS2007 (The 11th International Conference on Miniaturized Systems for Chemistry and Life Sciences), Paris 2007, ISBN 978-0-9798064-0-7, Publisher CBMS, Cat Nb 07CBMS-0001 and could be easily adapted to be performed within microchannels according to the invention.

In other exemplary embodiments, captured analytes may be released by deactivating the means for capture, and thus collected in another area of the microfluidic device, or in an external vial, for subsequent analysis of their content.

In other exemplary embodiments, cells captured in the active area of microchannels according to the invention may be cultured, and screened for some biological properties, such as proliferative power, genotype, phenotype, caryotype, response to a drug, a toxic agent, or a chemical. This may be advantageous for rare cells, for which such culture was not possible with methods of the art.

Other exemplary embodiments of the invention provide a method for diagnosis or prognosis or drug screening or drug discovery, or biotechnology applications, or stem cells selection, involving the capture and cultivation of cells in a microfluidic device, wherein said cells are present in a sample flown in said microfluidic device at a concentration smaller than 10 cells per microliter, smaller than one cell per microliter, or smaller than one cell per 10 μL, or smaller than one cell per 100 μL, or even smaller than one cell per ml.

Other exemplary embodiments of the invention provide a method for diagnosis or prognosis or drug screening or drug discovery, or biotechnology applications, or stem cells selection, involving the capture of at least one cell in a microfluidic device, being optionally any above-defined microfluidic device, wherein said at least one cell is present in a sample extracted from an animal or a plant, said sample is flown in said microfluidic device, and said captured at least one cell is cultured.

In a first family of embodiments, said cultivation is performed at the site within said microfluidic device where said cell is captured. In a second family of embodiments, said cultivation is performed ex situ, which is facilitated first by the small volume of the capture zone, and second by the de-activable nature of the capture elements.

Other exemplary embodiments of the invention provide a method for the capture of analytes, and particularly of cells or organelles, comprising a microfluidic device, being optionally any above-defined microfluidic device, comprising at least one microchannel, said microchannel containing a physically reversible array of self-assembled colloidal particles, wherein the sample containing said analytes is flown in said microfluidic device at a flow rate of at least 20 μL/hour, for example 50 μL/hour, 100 μL/hour, 200 μL/hour, 500 μL/hour, 1 mL/hour, 2 mL/hour, and up to more than 5 mL/hour.

Other exemplary embodiments of the invention provide a method for capturing, analyzing, cultivating, preparing, sorting or studying analytes,

wherein at least two populations of capture colloidal objects, with well distinct sizes or well distinct magnetization are flown in a microchannel, optionally of a microfluidic device as defined above,
at least one of said two populations of capture colloidal objects being flown in said microchannel in the absence of said analytes, and
at least one of said populations of capture colloidal objects carrying ligands for said analytes.

Any above-defined method may contain a step of releasing analytes from said active zone, and a step of analyzing, cultivating, or differentiating said analytes in at least one second analysis zone.

Both populations of capture colloidal objects may be flown in the microchannel in the absence of said analytes,

In some exemplary embodiments, both populations may be flown together, and in other exemplary embodiments, they may be flown separately. In the latter case, the larger particles are for example flown before the smaller ones

By “two populations of capture colloidal objects with well distinct sizes”, we mean that they have a combined polydispersity larger than 2, and in particular larger than 5, and even more larger than 10.

In another exemplary embodiment, the two populations of capture colloidal objects have a joint size distribution which is bimodal, one of the peaks of the distribution corresponding to a first type of capture colloidal objects, and the other peak corresponding to the second type of capture colloidal objects.

Other exemplary embodiments of the invention provide methods to perform activable, in particular reversibly activable, means to capture analyte colloidal objects and analytes.

For instance, if capture elements according to the invention are magnetic domains or electrically conducting domains, they may be activated by the application of an external magnetic or electric field, and induce the self-assembly of magnetic or dielectric capture colloidal objects, respectively, onto said capture elements.

Said capture colloidal objects may carry on their surface ligands for the analytes, and once self-assembled, may be able to capture said analytes, even if the activable capture elements themselves are unable to capture said analytes directly, which may provide several advantages, when combined with other aspects of the invention, such as the possibility of accommodating samples of large volumes on small footprints, not available in prior art.

In particular, one does not need to functionalize each microdevice individually with ligands, and a large volume of capture colloidal objects can be functionalized in a single step out of the microfluidic device of the invention, and a single batch preparation said large volume of capture colloidal object can be used to operate tens, hundreds or even thousands of Microdevices according to the invention.

As another advantage, this allows to switch on and of the capture of said capture colloidal objects by externally activable and de-activable means, in order e.g. to refresh the microfluidic channel after use, or in order to recollect without damage the captured analytes for further study or culture.

Other exemplary embodiments of the invention provide a method for the sorting, analysis, typing or culturing of analytes, notably cells, wherein a sample containing said analytes is first flown in the active zone of a microfluidic device, being optionally any microfluidic device as above-defined, aliquots of reagents are subsequently flown in said active zone, and wherein the ratio of the initial volume of sample containing said analytes, to the volume of at least one reagent aliquot, and preferably the volume of all reagents aliquots, used for sorting, typing, or analyzing said analytes is at least 10, preferably at least 50, 100, 200, 500, or 1000.

Other exemplary embodiments of the invention provide a method for the capture, culture or sorting of analytes, and notably of rare cells, said method comprising a first step of providing a blood sample of volume A, a second step of lysing red blood cells from said sample, a third step of resuspending nucleated cells from said sample in a volume B, and a 4^{th} step of sorting a subset of nucleated cells from said volume B in a microfluidic device, being optionally any microfluidic device as above-defined, wherein said volume B is less than 3 times, preferably less than 5 times, yet preferably less than 10 times, 20 times, 50 times or even 100 times smaller than said volume A.

Other exemplary embodiments of the invention provide a method for the capture of rare cells, comprising a first step of providing a first blood sample of volume A, and at least a second step of flowing said sample or a pretreated sample obtained from said first blood sample in an active zone or a combination of active zones of a microfluidic device, being optionally any above-defined microfluidic device, where said rare cells are captured, wherein said flowing step lasts less than two hours, preferably less than 1 hour, and even more preferably less than ½ hour, and wherein in less than 1 hour, with a ratio between the initial sample volume A to the volume of the active zone or the combined volume of the active zones in which said cells are captured is larger than 100, preferably larger than 500, 1000, 2000, 5000, 10 000, and in particularly optimized cases even up to 100 000.

Other exemplary embodiments of the invention provide a method for magnetic capture of cells or analytes from an initial raw sample with a microfluidic device optionally anuve above-defined microfluidic device, wherein the total mass of magnetic particles used for treating least 1 mL, and preferably at least 5, 10, 20 and up to 50 mL of raw sample is less than 10 mg, preferably less than 5 mg, 2 mg, 1 mg, 0.5 mg, 0.2 mg, or less than 100 μg.

Other exemplary embodiments of the invention provide a method for the sorting or the analysis, or a combination of sorting and analysis, of analytes and notably cells, with a microfluidic device, being optionally any above-defined microfluidic device, comprising the steps of
capturing said analytes with magnetic particles
imaging or analyzing said analytes
extracting from said image or from the data resulting from said analysis at least one quantitative numerical result regarding at least one predefined criterion, said extraction being performed for at least one analyte,
comparing said at least quantitative numerical result with a reference value.

Another advantage of the invention, which will be more apparent upon the description of some preferred embodiments, is a strong reduction of reagents volumes as compared with state of the art. Since these reagents often contain biological material such as antibodies, or chemicals such as micro or nanoparticles or fluorescent dyes, and such materials are often very expensive, or available in limited quantities, this advantage is a considerable one. Typically, a volume of reagents equal to a few times the combined volume of the active zone is sufficient for treating the captured analytes, so the invention may be implemented with success with aliquots of reagents smaller than 1 mL, preferably smaller than 500 smaller than 200 μL, smaller than 100 μL, and sometimes smaller than 50 μL, for initial sample volumes as large as 1 mL, preferably as large as 2 mL, 5 mL, or even 10 mL. Typically, then the ratio of the initial volume of sample containing said analytes flown in the active zone, to the volume of at least one reagent aliquot flown in said active zone, and preferably the volume of all said reagents aliquots, used for sorting, typing, or analyzing said analytes is at least 10, preferably at least 50, 100, 200, 500, or 1000.

Other examples of some preferred embodiments of the invention provide a method for the capture and optical analysis of cells, wherein cells are captured, and submitted to optical analysis in the same microfluidic chip, being optionally as defined above, or in the same microchannel.

Other exemplary embodiments of the invention provide a method for the capture of cells and for the molecular analysis of their content, wherein said capture and said molecular analysis are performed in the same microfluidic chip, or in the same microchannel.

In another of its aspects that will be more apparent from the embodiments described in part 9/below, another objet of the invention is to provide an instrument comprising at least one active zone in which analytes can be sorted, analysed, typed or cultured, and additionally comprising means to activate a magnetic field in said active zone, wherein said means involve the translation of permanent magnets, and wherein said translation induces a change in the amplitude of said magnetic field in said active zone without changing significantly its direction or its homogeneity

Other exemplary embodiments of the invention provide an instrument comprising an active zone in which analytes can be sorted, analysed, typed or cultured, said active zone being optionally part of a microfluidic device, in particular of any above-defined microfluidic device comprising means to activate a magnetic field in said active zone, wherein said means involve the translation of permanent magnets, and wherein said translation induces a change in the amplitude of said magnetic field in said, active zone without changing significantly its direction or its homogeneity.

Other exemplary embodiments of the invention provide a microfluidic device, optionally as defined above, comprising an active zone or a combination of active zones in which analytes can be captured, sorted, analyzed, typed or cultivated, wherein the volume of said active zone or combination of active zones is smaller than 50 μL, preferably smaller than 20 μL, 10 μL, 5 μL, 2 μL or 1 μL, and wherein liquid can be flown in said active zone at a flow rate of at least 100 μL/hour, 200 μL/hour, 500 μL/hour, 1 mL/hour, 2 mL/hour, and up to more than 5 mL/hour, without exceeding an average flow velocity of 1 mm/second, preferably 800 μm/s, or 200 μm/s, or being around 100 μm/s.

The average thickness of said active zone or combination of active zones may be smaller than 200 μm, preferably smaller than 100 μm, and notably comprised between 30 μm and 100 μm, and preferably between 40 μm and 80 μm, even more preferably between 50 μm and 70 μm.

Such a microfluidic device may be part of a unit further comprising at least one any above-defined microfluidic device.

In all the above-mentioned exemplary embodiments, the microfluidic device or the instrument may comprise a second analysis zone and means to transport the analytes from the active zone to the analysis zone.

The second analysis zone may be comprised in the same microfluidic device as the active zone and said means to transport may be microfluidic means.

Other characteristics and advantages of the present invention appear on reading the following detailed description of non-limiting embodiments, and on examining the accompanying drawings, in which:

### FIGURES

FIG. 1 represents a general layout for microfluidic and imaging system for the application of the invention,

FIG. 2 represents a first way of creating magnetic domains for the application of the invention, based on microcontact stamping,

FIG. 3 represents another way of creating magnetic domains for the application of the invention, based on convective self-assembly,

FIG. 4 represent different possible arrangements of magnetic domains suitable for implementing the invention,

FIG. 5 represents examples of layouts of microchannels for the implementation of the invention,

FIG. 6 represents 3D views of embodiments of the invention optimized for small footprint,

FIG. 7 represents examples of simulations of flow in microchannels suitable for the implementation of the invention, and how this simulation can be used to improve the homogeneity of the flow,

FIG. 8 represents an example of a device for providing pulseless switching of flow within the invention,

FIG. 9 represents different ways to create switchable magnetic fields suitable for the invention,

FIG. 10 represents simulation of the macroscopic magnetic field switching in the embodiments of FIG. 9B,

FIG. 11 represents a numerical modelling of the local magnetic field in the vicinity of magnetic capture objects of the category of magnetic beads, after their self-assembly onto a magnetic capture element prepared by microcontact printing,

FIG. 12 provides examples of capture efficiency profiles in an embodiment of the invention,

FIG. 13 represents an example of phenotyping of cancer cells from small volume samples in the invention,

FIG. 14 represents an image of reconstitution of 3D images from confocal microscopy in the invention,

FIG. 15 represents examples of typing of breast cancer metastasis tumour cells in the invention and,

FIG. 16 represents an example of accelerated imaging within the invention by denoising software

### DETAILED DESCRIPTION OF SOME EXEMPLARY EMBODIMENTS AND EXAMPLES

1/: General layout

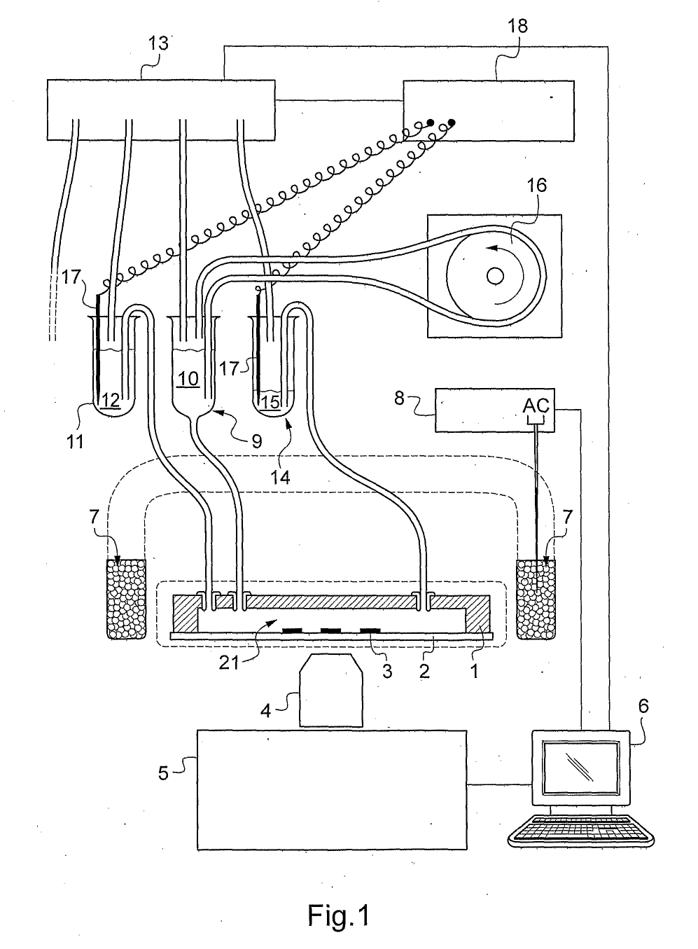

A general layout of a possible embodiment is described in FIG. 1 (not to scale).

It involves a microfluidic system, comprising a microchannel network 21 (shown in cut and simplified), enclosed between a microfluidic chip 1, and a window 2. The window carries on its face in contact with the microchannel, capture elements 3. Optionally, said window is located in front of a microscope objective 4, preferably with a high magnification, from 20× to 100× and a high numerical aperture. This objective is part of an imaging system 5.

Essentially all high quality microscopes available on the market (eg, as a non-limitative exemplary list, by companies as ZEISS®, LEICA®, OLYMPUS®, NIKON®) may be used for that purpose. Preferably, but not mandatorily, said microscope is an inverted microscope. Notably, too, the imaging system in the invention may also be a custom one, built for the purpose of the invention, and optimized for it. In some embodiments, said imaging system is able to perform 3D imaging, or optical sectioning. Particularly interesting are confocal microscopes, and microscopes based on a spinning disk system. In other embodiments, said imaging is a conventional microscope, preferably able to do automated scanning and positioning.

Connected to the microfluidic chip 1 are an inlet connected to one or several sample inlet vial 9, containing sample 10.

Optionally, the microfluidic chip is also connected to one or several outlets connected to one or several outlet vial(s) 14, for collection of fractions 15

Optionally, too, the microfluidic chip is connected with one or several buffer or reagents vials 11, containing buffers or reagents 12.

Here, vials are represented as separate elements. In some other embodiments, however, vials for samples reagents and fractions, may be integral to the chip, in order to get a more compact layout and minimize fluidic connections.

In the embodiment presented here, the flow of reagents, samples buffers, etc, is controlled by a pressure controller 13 such as the MFCS from FLUIGENT®, thanks to tubings relating said flow controller to the corresponding vials.

In other embodiments, flow may be created by Syringe pumps, as distributed e.g. by Harvard Instruments®, or CETONI®, or peristaltic pumps. In other embodiments, flow may be monitored by microfabricated pumps integral to the chip, as described e.g. in Unger et al., Science 2000, 288, 113-116).

The use of pressure controller, however, may be advantageous, because it avoids pulsing, which may hinder proper functioning of the invention.

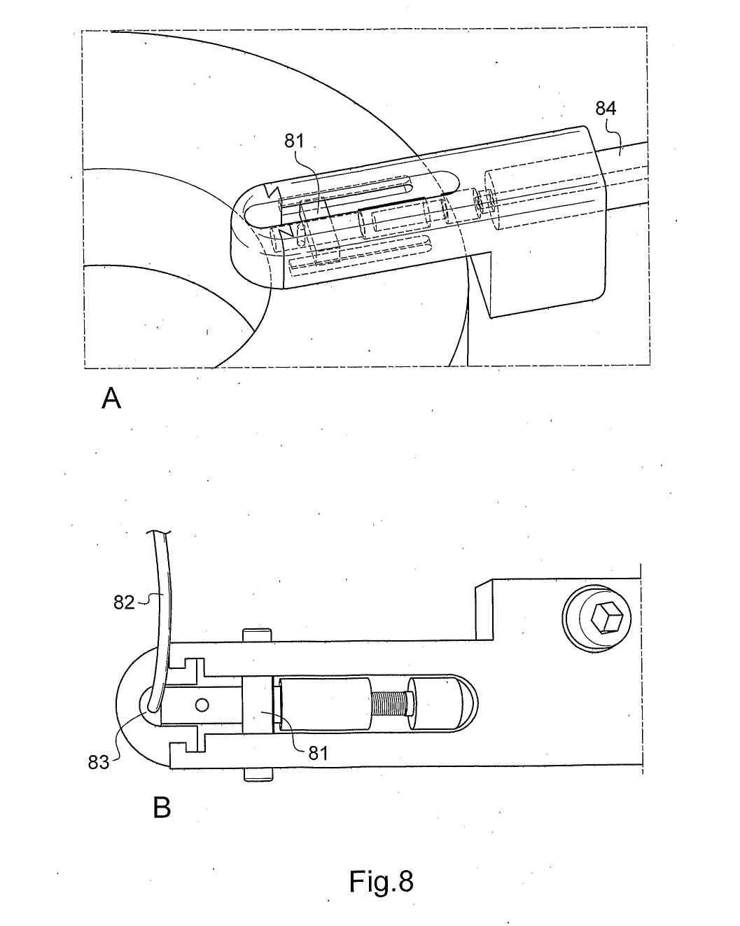

Optionally, too the tubings relating vials 9, 11, 14 to the chip 1, may comprise along their path additional valves (not represented in FIG. 1). Preferably, said valves are of the type with progressive closure and opening, as described in more detail in FIG. 8 and part 7/ below.

Back to FIG. 1, if the sample fluid contains elements that tend to sediment, such as e.g. cells, sample vial 10 may optionally comprise a mixing means 16, to prevent such sedimentation. Mixing means may be of different kinds, such as rotating the vial itself, or having in the vial a small magnetic agitator, or, as represented here, with a peristaltic pump 16 recirculating continuously or intermittently the sample fluid.

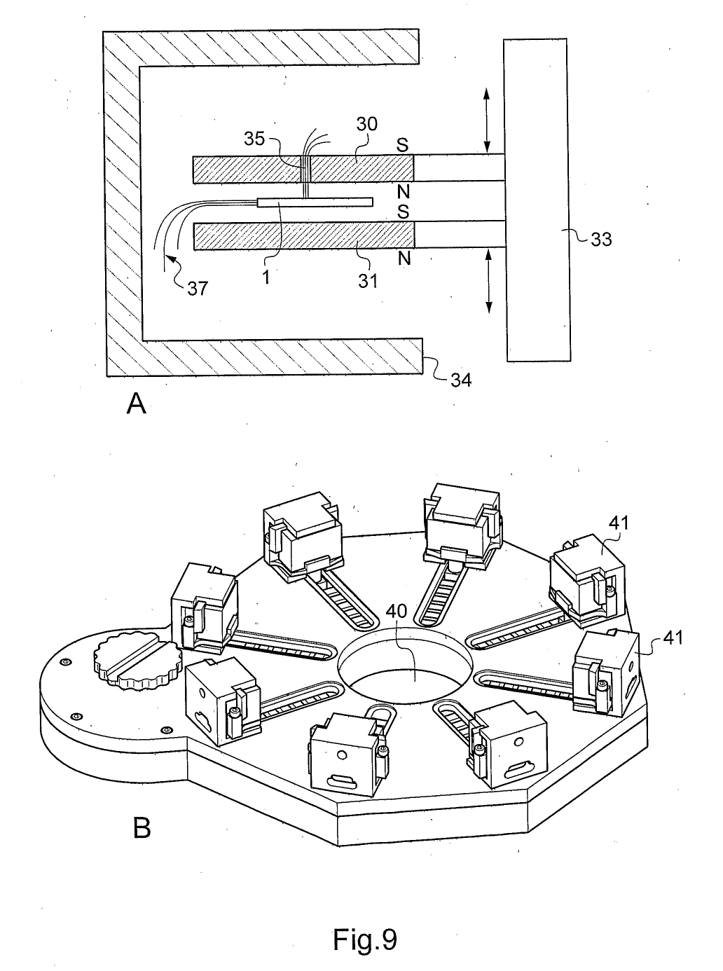

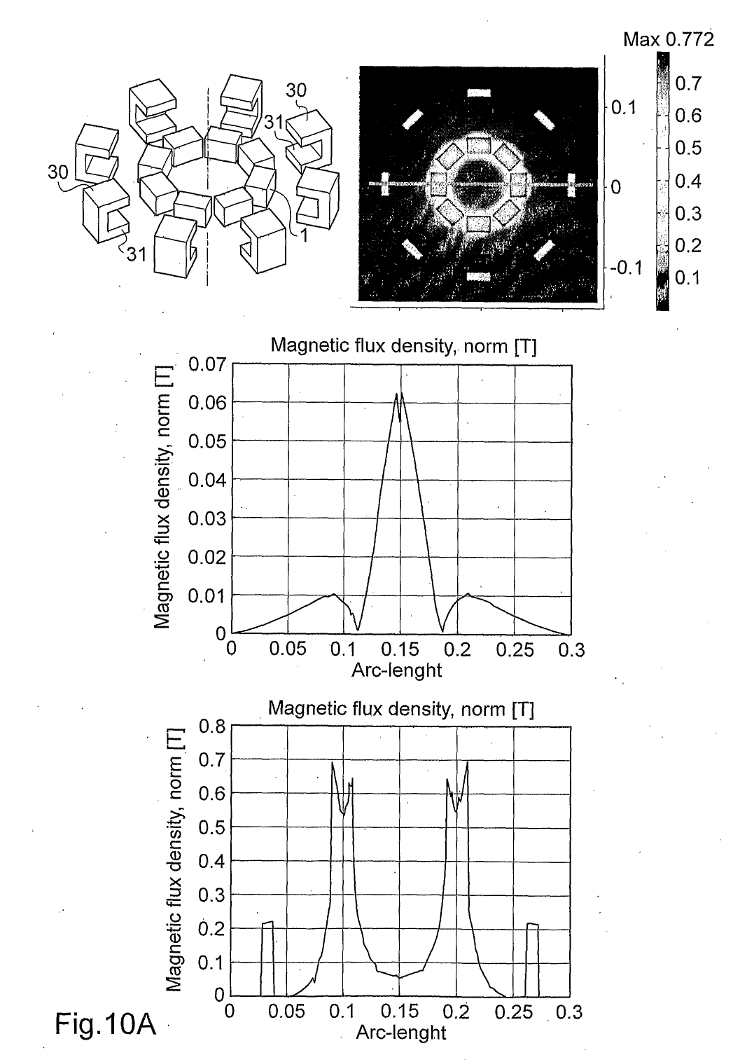

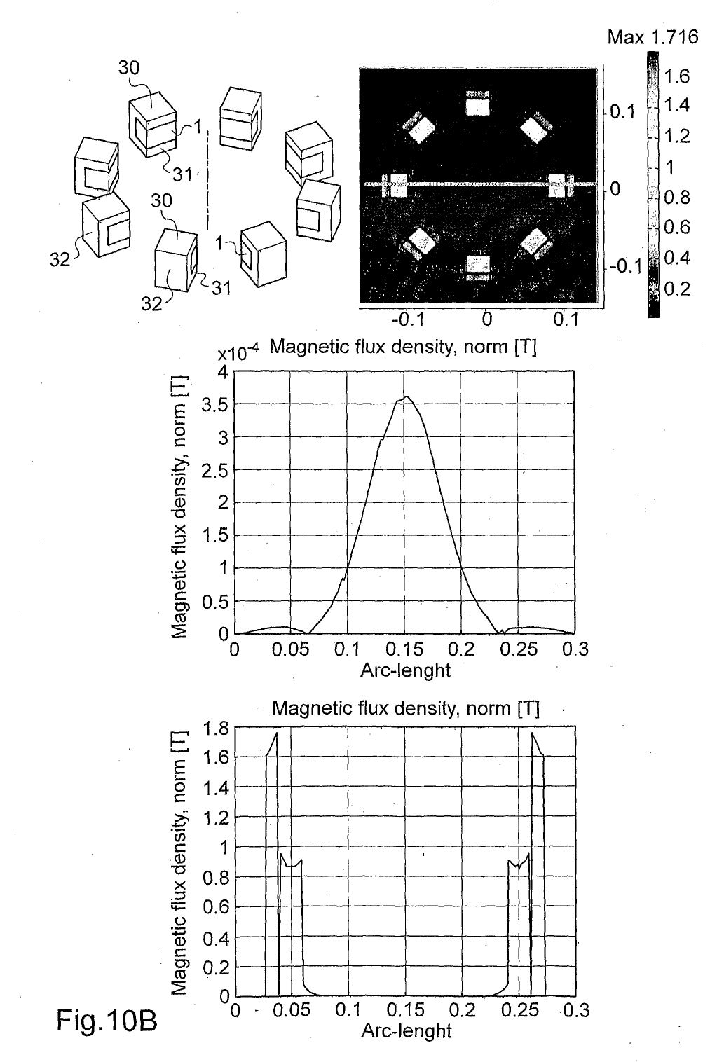

If the capture elements 3 are of the type of activable capture elements, the invention may optionally comprise means to activate them. For instance, if they are magnetic, the chip is advantageously placed inside a coil 7 (presented here as a cut), able to generate a magnetic field essentially perpendicular to the chip's plane, when receiving current from a current generator 8. The current delivered may be AC or DC. Other exemplary embodiments of components suitable for activating magnetic capture elements are shown in FIGS. 9 and 10

If capture elements are of the type of conductive activable elements, magnetic coil 7 and power supply 8 are not necessary. Instead, one should provide means to activate such conducting elements. This may be achieved, e.g. by inducing in microchannel 21a longitudinal electric field thanks to electrodes 17 placed in at least one of each vials 11 and 14, and connected to a voltage generator, preferably a high voltage generator, such as LABSMITH® high voltage generator, Trek® 10 kV, Trek® 20 kV, or EMCO®“OctoChannel”. Notably, using high voltage generators, caution should be taken according to the rules for high voltage manipulation, involving security emergency stop, enclosing all elements in electric or fluidic contact with the electrodes in a cabinet connected to the generator emergency high voltage cutoff entry.

By activating voltage generator, field lines traveling in the fluid in the microchannel are attracted by the capture elements 3 of electrode-type in this example, which are more conductive than the fluid, thereby creating electric field gradients. These gradients are able to attract, by dielectrophoresis, dielectric particles presenting with the fluid contained in the microchannel a contrast of complex dielectric constant. Preferably, said dielectric particles bear ligands for analytes of interest.

Optionally some or all of electric and electronic devices associated with the invention may be controlled by a computer, or an electronic device. Optionally, said computer is the same computer 6 as used for image analysis, but it may be another too.

### 2/ Preparation of an Array of Capture Elements of the Magnetic Type, for the Activable Capture of Capture Objects of Magnetic Type, by Microcontact Stamping

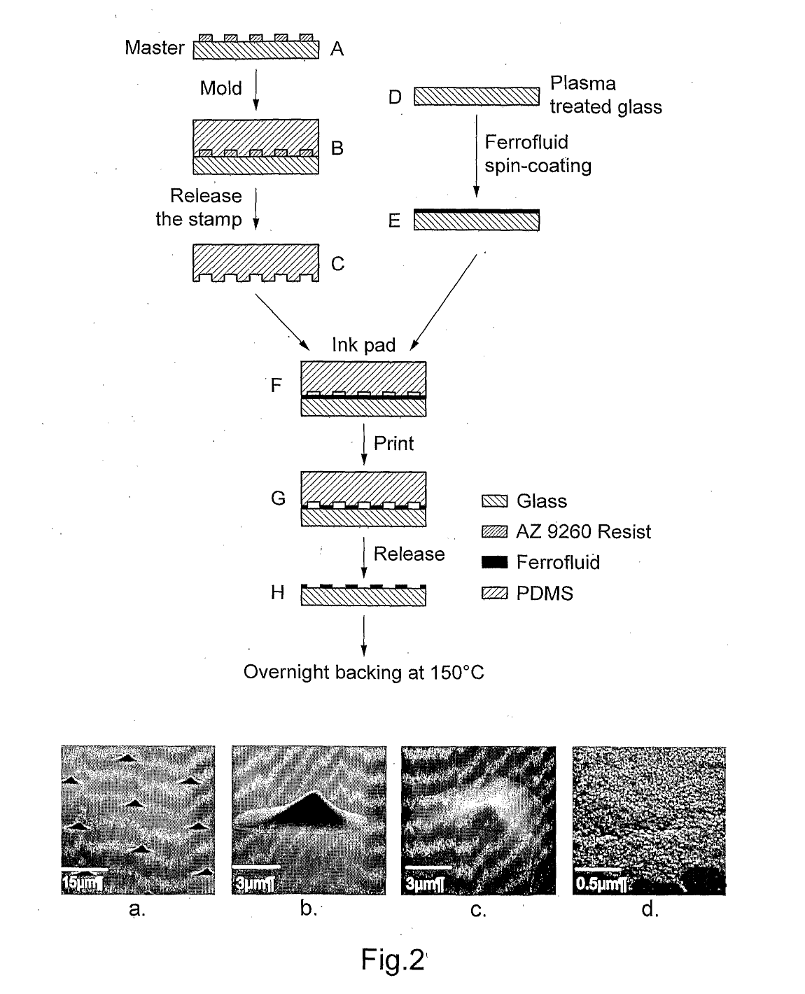

FIG. 2 displays the flow stream of a method, by which capture elements of the magnetic type may be prepared, by reference to part 1/:
in FIG. 2 A, a glass master bearing a patterned photoresist layer corresponding to the negative of the desired magnetic structures is prepared by conventional photolithography;
in FIGS. 2 B and C, PDMS (polydimethylsiloscone) is cast on this master and peeled, forming the inking stamp;
in FIG. 2 D, a second glass plate is cleaned with oxygen plasma,
in FIG. 2 E, said second glass plate is covered with a thin film of ferrofluid ink by spin coating;
in FIG. 2 F, the stamp is contacted with the ink pad to collect magnetic ink on its posts,
in FIG. 2 G the stamp is pressed against a coverslip by manual or mechanical means, to transfer onto it the magnetic pattern; and
in FIG. 2 H, the coverslip is baked overnight.

FIGS. 2Ia, 2Ib, 2Ic and 2Id, represent views, obtained with Scanning Electron Microscopy at different scales, of the hexagonal array of magnetic domains obtained this way.

Several methods for microfabricating magnetic patterns had been previously proposed. Nickel (Inglis D W, Riehm R, Austin R H, Sturm J C (2004) J Appl Phys 85:5093-5095) or cobalt (Yellen B, Friedman G, Feinerman A (2003) J Appl Phys 93:7331-7333) template may be obtained by standard lift-off process. Ni pattern may also be generated by electroplating (Guo S S, Zuo C C, Huang W H, Peroz C, Chen Y (2006) Microelec Eng 83:1655-1659). These techniques require advanced equipment and clean-room facilities. A “soft lithography approach, consisting in encapsulating magnetic beads in PEG after UV photopolymerisation, was also proposed (Pregibon D C, Toner M, Doyle P S (2006) Langmuir 22:5122-5128), but this requires an upstream step for organizing magnetic beads on the surface. A new and particularly simple method is proposed here, as summarized in FIG. 2, for preparing magnetic patterns, based on the micro-contact printing of a water-based ferrofluid (“magnetic ink”) onto glass, and fixation by a post-bake thermal treatment.

A mask bearing a 40 μm hexagonal pattern of 10 μm dots was designed using Qcad software and printed at a resolution of 24.000 dpi (SELBA®, Switzerland) on a polyethylene terephtalate (PET) film. The mask features were transferred in a positive resist AZ9260 (MICROCHEMICALS®, Germany) spin-coated on a glass substrate, forming a master with holes of 10 μm in diameter and 8 μm in height. A stamp was formed by preparing a PDMS replica of the master: PDMS (SYLGARD® 184, Dow Corning, France) was mixed at a 1:10 base:curing agent and cured 3 h at 65° C. before being peeled. An “ink pad” was prepared by spin-coating magnetic ink (water based ferrofluid M3300, LIQUIDS RESEARCH®, UK) on a glass slide, previously washed with acetone. The stamp was contacted with the ink pad and released. The magnetic ink was then transferred onto a naked glass slide (coverslip) by conformal microcontact stamping. After stamping, the stamp was for example immediately wiped with isopropanol, allowing numerous re-uses. The glass slide bearing the magnetic patterned was baked overnight at 150° C.

Optical imaging shows that a regular, uniform and essentially defect-less array was achieved over the whole surface (FIG. 2Ia). A characterization of the dots by electronic microscopy shows that they adopt a rather reproducible cone like shape (see FIG. 2Ib and 2Ic). This feature is favourable for a better centering of the magnetic column on the spot's center. The diameter of the dots, 5+/−1 μm, is significantly smaller than the initial size of the pins in the stamp. No ferrofluid is found between the dots. Atomic Force Microscopy (AFM) measurement (data not shown) indicates that the top of the spot is 500+/−50 nm high. A freshly prepared array does not resist to the application of flows in a biological buffer, but after baking overnight at 150° C. yields the array may be washed and used repetitively for days. Baking may lead to a fusion of the polymer layers surrounding the magnetic particles present in the ferrofluid, into a glassy hydrophobic bulk polymer material, following a process similar to that at play in thermo-setting paints.

3/ Method for Preparing an Array of Capture Elements of the Magnetic Type, for Implementation of the Invention, Said Method being Based on Convective Self-Assembly

Microcontact printing, as described above, is known a versatile and rapid technique for surface patterning. Nevertheless this technique suffers from its poor applicability to highly viscous inks. In particular, in the case of ferrofluid printing, experiments show a clear lack of reproducibility and a poor spatial resolution of this technique, if high care is not exerted during the printing process, especially. To overcome these limitations while keeping a parallel and low cost patterning technique a self assembly technique is proposed to directly integrate magnetic particle on a surface. In this approach particles are used as building block to create magnetic patterns on a surface that may be further used as anchor point for the assembly of magnetic columns.

Self-assembly is defined as the autonomous organization of objects into ordered structures. It is one of the most efficient approaches to order large numbers of small objects on surfaces. The resulting structures, however, are often limited to certain dense packings, whereas the placement of individual objects through such method is usually difficult. Techniques combining self-assembly with topographical patterning of the substrate are well-suited to address this limitation.

In a particularly interested embodiment of the invention, convective self assembly is used to assemble magnetic beads on a surface, in order to guide the subsequent assembly of magnetic capture elements under a magnetic field.

In this approach, capillary forces are used to direct the organization of particles on a patterned surface (such as a patterned PDMS surface). It is based on the confinement of particles induced at the three-phase contact line of a droplet that is dragged over a substrate. A droplet of a colloidal suspension of magnetic particle is pinned between a fixed confinement slide and a moving substrate. The capillary force induced close to the contact line induces their immobilization in the recessed areas of the substrate while no deposition occurs on the flat areas.

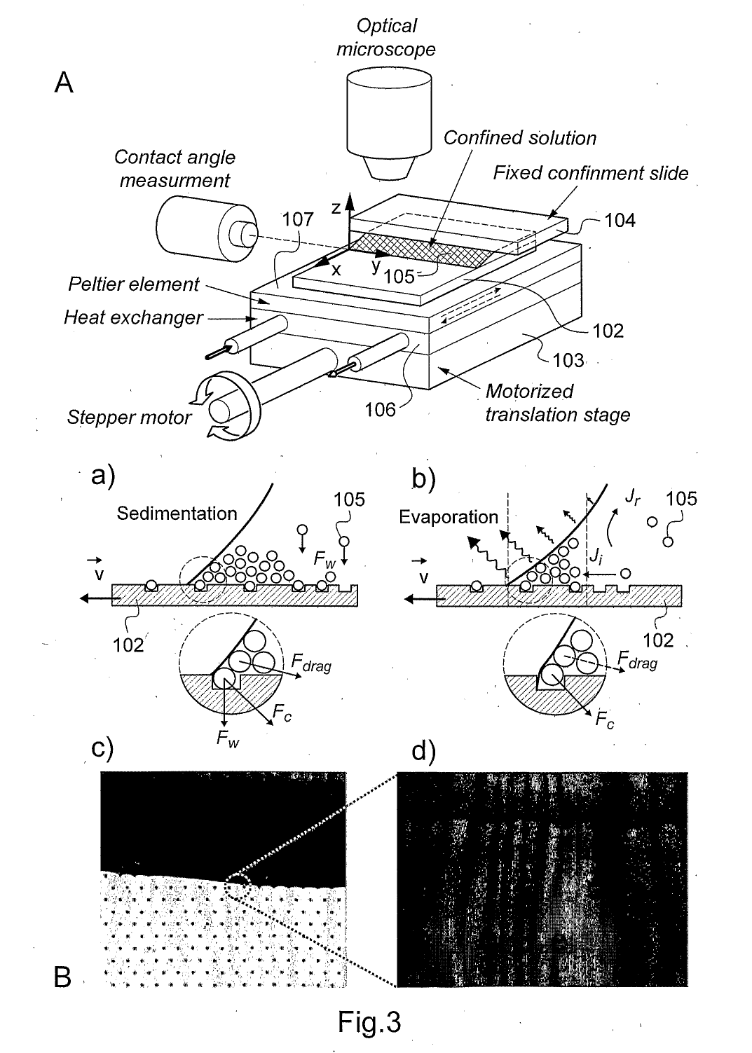

Accumulation of particles close to the contact line region is required to initiate the assembly process. First the local increase of the local particle concentration is necessary to maximize probability of particle trapping in the structures. Second, in the case of Brownian particle, this accumulation helps in reducing the self-diffusion of particles and thus promotes their immobilization on the surface. In both cases, this mechanism may be easily controlled by tuning experimental parameters such as particle solid content or substrate temperature. In this latter case, the evaporation of solvent close to the contact line will induce a pre-concentration mechanism of brownian particles from the bulk suspension towards the contact line (see FIGS. 3Ba) and 3Bb)). In the case of heavy particles which are sedimenting, this accumulation process is induces by the dragging forces exerted by the meniscus while moving over the substrate (FIG. 3Bb)).

A device 100 suitable for this convective self-assembly is schematically represented in FIG. 3A. The substrate 102 on which the capture elements 3 are to be assembled (here a glass coverslip bearing a thin layer of PDMS with microfabricated holes, is positioned on a motorized platform 103 with temperature control and a tilted glass slide 104 (typical angle, 5°) is positioned at about 1 mm above the PDMS upper surface. The temperature control is for example achieved thanks to a heat-exchanger 106 and/or a Peltier element 107. A droplet containing magnetic beads 105 (e.g. Dynabeads 4.5 μm) in a buffer added with surfactant (an example of composition: PBS 0.1%, Triton X45, SDS 0.01 M, for Dynabeads and PDMS: the solution composition may be adapted depending on the hydrophobicity of the beads and on the structured surface) is deposited between the glass slide and the PDMS. The slide is then translated parallel to the structured PDMS surface, in order to create a recessing meniscus (see FIG. 3Ba) and 3Bb)). The speed may also be adapted depending on the beads, solution and substrate. For The above solution and PDMS surface, 20 μm/s is a good value.

An exemplary experiment with 4.5 μm magnetic particles (DYNAL®) assembled on a patterned PDMS surface (FIGS. 3Bc) and 3Bd)) show the results obtained at an assembly speed up to 100 μm.s. These particles could be efficiently assembled on a 4×4 cm² substrate in less than 7 min. FIG. 3Bd) shows a high magnification picture, with contact line on the top, and 12 microfabricated holes, and a single bead in each of them. By tuning the size of the hole, different number of the beads can be assembled. Using structures for single particle trapping (diameter 5 μm, depth 4 μm), an assembly efficiency, i.e the number of immobilized patterns/number of immobilization sites, was measured around 98.5% (not shown). FIG. 3Bc) shows a lower resolution image, showing the high quality and regularity of the assembly.

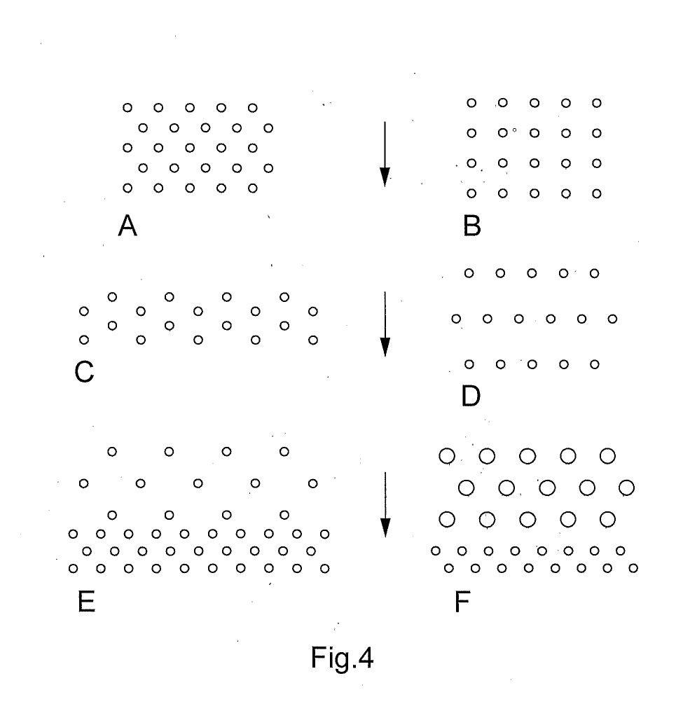

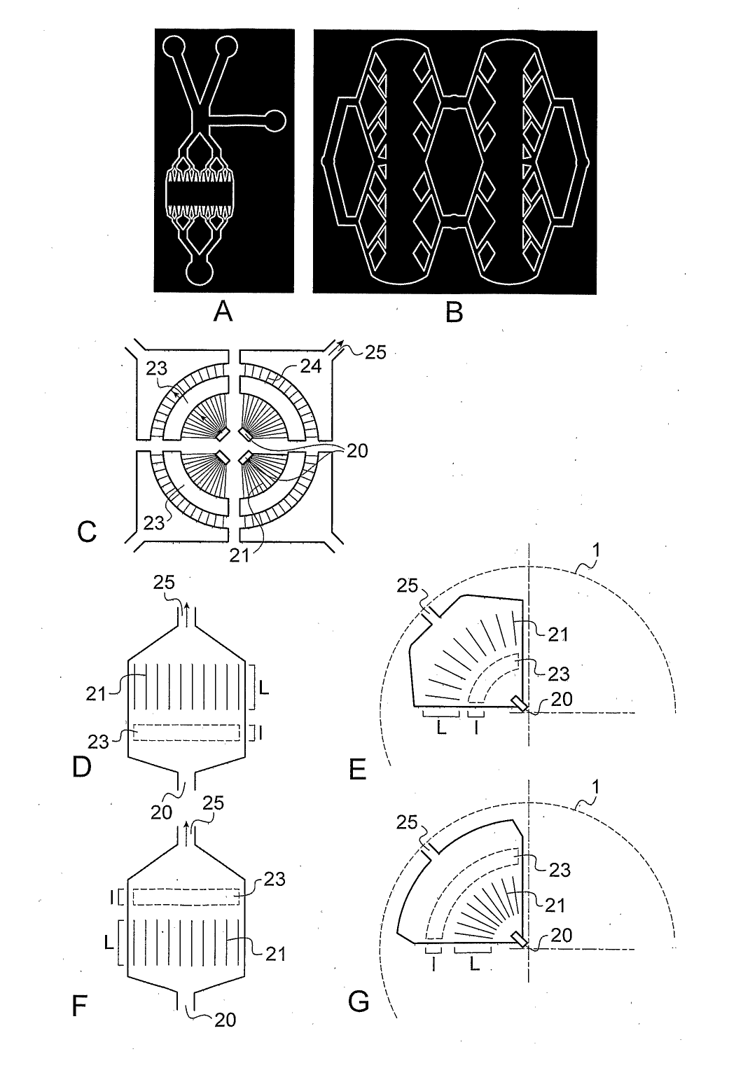

In FIG. 3, the array is an hexagonal array. However, other types of arrays, such as square (as in FIG. 4B), parallelepipedal, or essentially any kind of periodic array can be used in the invention depending of the size, distribution of size, concentration, shape, of the analytes to be captured. Also, arrays can be designed by starting from a given array, and deforming it by reducing its size homotetically in the direction of flow, as in FIG. 4C, or oppositely in the direction perpendicular to flow, as in FIG. 4D. Also, different geometries and spacings between capture domains can be combined in a single active zone, as exemplified in FIG. 4E or 4F. Said implementation is notably interesting when the analytes to be separated have a range of sizes, or when it is interesting to separate analytes but their size. In other embodiments, finally, it may be interested to dispose capture elements in a non periodic array.

### 4/ Example of Layouts of Microchannels Suitable for Defining Active Zones According to the Invention, and for Flowing Samples and Reagents in Said Active Zones

Numerous layouts for microchannels 21 within the invention may be used, and some embodiments are proposed in FIG. 5. Typically, the aim of the layout is to provide means to flow capture objects, such as magnetic particles, samples, reagents, rinsing solutions, in the active zone, and optionally to release and collect the captured analytes.

FIGS. 5 A and B provide examples in which the active zone is parallelepipedal. Notably, within the invention, it is interesting to distribute fluids through a “delta” configuration, so that the flow is distributed evenly all across the active zone (more details about ways to accomplish this will be provided below). In both FIGS. 5A and 5B, the capture zone has a width perpendicular to flow direction, larger than its length in the flow direction. In FIG. 5A the width over length ratio is about 3.5, in FIG. 5B the device involves two capture zones with a ratio of the combined width to the length of about 17. The width is defined as perpendicular to the flow (which means horizontal, i.e. parallel to the shortest side of the paper, in FIG. 5A and vertical, i.e. parallel to the longest side of the paper, in FIG. 5B), and the length along the flow direction (which means vertical in FIG. 5A, and horizontal in FIG. 5B).

Other types of layouts are provided in FIG. 5C FIG. 5C displays a system in which a radial disposition is used to keep the area occupied by the system limited, and the flow uniform across the active zone. In this case, the active zones are inscribed along a circle. This layout shows that one can also combine in a single chip several (here four chips) different active zones with independent inlets and outlets. Typically, these chip designs comprise one or several inlets 20, distributing flow evenly towards one or several active zones 23, through an array of distribution microchannels 21. Flow is then directed towards an outlet 25 by a collection of microchannels 21.

Yet other types of layouts are displayed in FIG. 5 D to G: They comprise an inlet 20, distributing fluid to an active zone 23, through and array of microchannels 21, towards an outlet 25. In this case, flow uniformity in the active zone 23 is achieved by keeping the length L of the microchannels significantly larger than the length l of the active zone, typically at least 5 times larger, 10 times larger, 20 times larger, or up to 50 times larger.

All these different layouts are represented in a sketchy way, and those skilled of the art know how to optimize design, e.g. regarding uniformity of flow, notably using hydrodynamic simulations. An example is given below

### 5/ Example of Optimization Process for Improving Flow Uniformity in the Active Zone in a Microfluidic Layout of the Inv

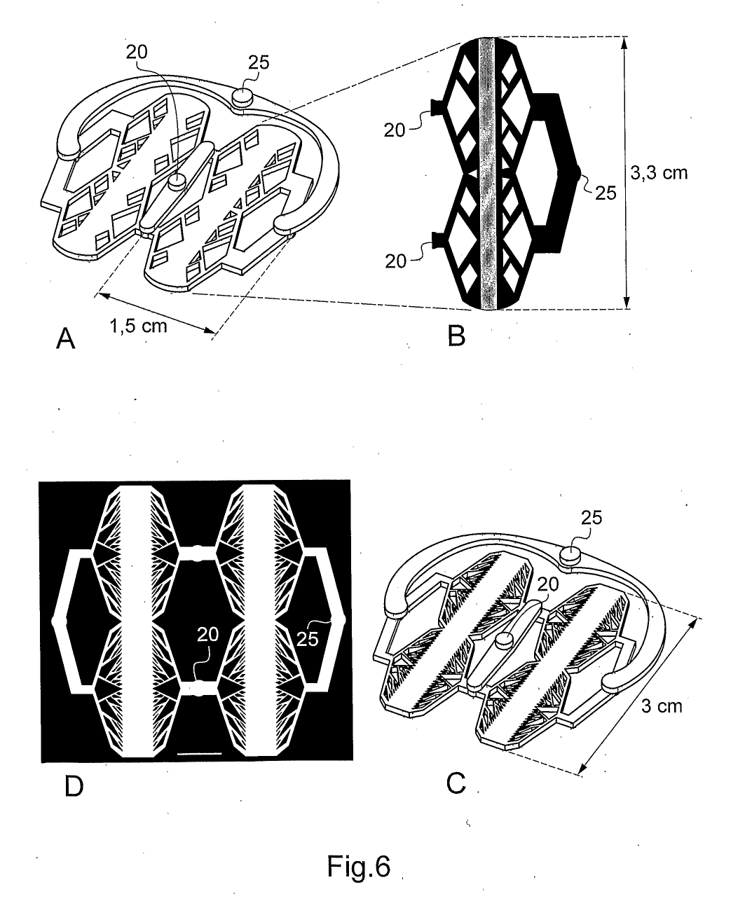

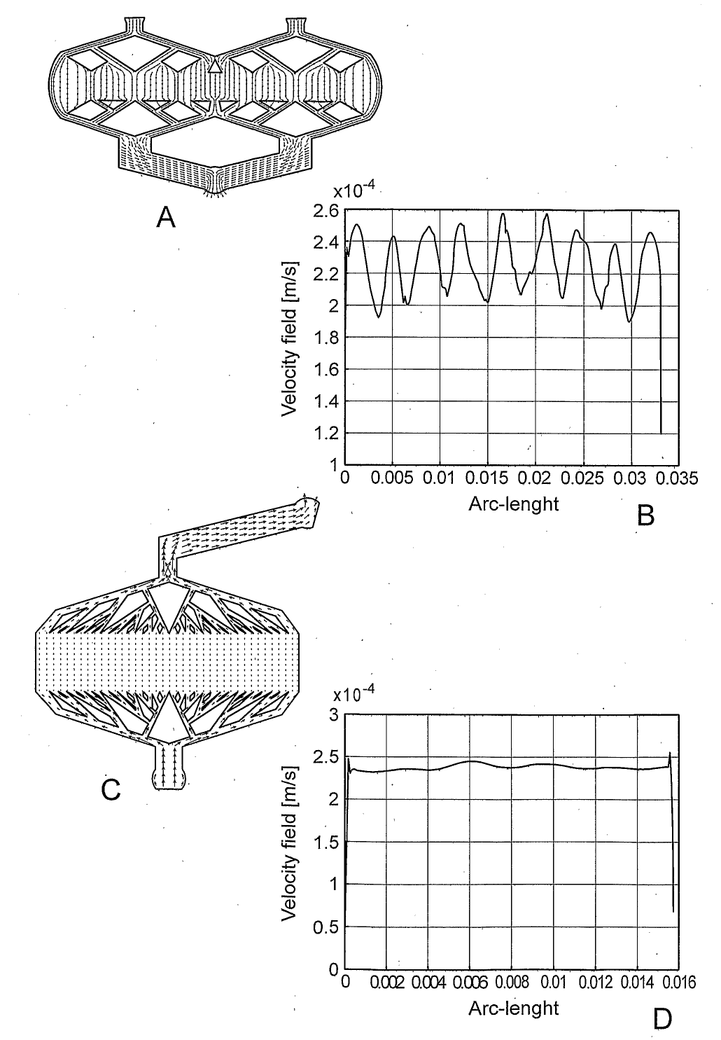

FIGS. 6A to 6D show two generations of layouts of the type describe in FIG. 5B, both in 2D and 3D: The 3D image shows a way to distribute fluids in a second fluidic layer, in order to keep the footprint minimal. FIGS. 7A and B, respectively, display the flow vectors and the distribution of flow velocity in the middle of the length of the active zone, across the active zone width, for a layout comparable to that of FIG. 6A. These data were obtained by COMSOL simulation.

With the software COMSOL 3.4, hydrodynamic flows were simulated in the geometry represented on FIG. 1.b. with a thickness 50 μm and the following boundary conditions:

Input speed: 1.3 mm/s

Output pressure: 0 Pa

No-slip boundary condition over all other walls.

The Reynolds number in the micro system being weak (Re=0.1), the used model is Stokes flow governs by:

$$ρ \frac{\partial u}{\partial t} - V \cdot [ - pI + η ( Vu + {( Vu )}^{T} ) ] = F$$

$$V \cdot u = 0$$

Where
ρ is the fluid's density (kg/m3)
u represents the velocity vector (m/s)
p equals the pressure (Pa)
η denotes the dynamic viscosity (Pass)
F is a body force term (Pa·s)
I is the identity matrix

In the following simulations, density ρ is equal to 1000 kg/m̂3 et the viscosity θ is equal to 10̂(−3)Pa·s.

FIGS. 7 C and D show the same, for an improved design, with a more fine distribution of distribution and collection microchannels: In 7B, the fluctuations represent about 20% of the average value, whereas in 7D, they only represent less than 5%.

### 5/ Examples of Fabrication Method for Microchannels and Active Zones

Once the layout of microchannels 21 and active zones 23 have been designed, microchannel arrays must be fabricated, and closed by a substrate, typically a window 2. The window 2 may comprise capture elements 3 prepared e.g. according to parts 2/ or 3/ above. Alternately, in some embodiments the capture elements 3 can be on the side of the active zone 23 opposite to the window. As an example, said microchannel array 21 may be made of PDMS. A protocol for preparing such microchannel array, of the type described in FIG. 6 with two microchannels layers, is as follows:

### Master Fabrication

In order to fabricate a microfluidic chip 1, it is first necessary to prepare a mould on glass or on silicon. Briefly, the stages consist in conceiving a mask on which are printed the design of the microfluidic channels. These patterns are transferred on a photo-sensitive resist (SU8 resist from Microchem or SY resist from Elga Europe) by exposure to UV-light (Suss Mask aligner). Beforehand, this resist has been spread using a spin-coater in a fine layer over a glass or silicon substrate, with a thickness which determines that of the microfluidic channels. The motives are finally developed in reagents suited to each sort of resist. A fine coating of silane is deposited on its surface at the end of process to avoid that the PDMS adheres to the surface of the mould during the subsequent mouldings.

### PDMS Replica

A 10:1 mixture of Poly-DiMéthylSiloxane (PDMS) Sylgard 184 silicone elastomer and curing agent (Dow Corning) is poured over the wafer to form a 5 mm thick layer and cured at 65° C. for 2 h. The PDMS channels are then peeled off the wafer, reservoir holes of 2 mm are punched with flat-end needle. PDMS surface is cleaned with isopropanol, dried with air, treated in an air plasma for 30 s (to activate its surface) and is irreversibly sealed on a substrate.

### Chip Assembly

The design of our chip is, in exemplary embodiments, constituted of three superimposed PDMS-layers: two layers of microfluidic channels and a layer with a magnetic pattern which controls the geometry of magnetic columns array and enhances its stability. Both microfluidic layers are first punched and stuck together after air plasma treatment. The microfluidic chip is then sealed over the bottom-PDMS layer bearing magnetic particles, as described below.
In order to avoid particles or cell adhesion on PDMS walls, some PDMA-AGE [Chiari, electrophoresis, 2000] is introduced into the channels immediately after the chip-sealing during 1 h and then washed with PBS+0.5% BSA (Bovine Serum Albumin, Sigma).
6: Microchannel Arrays with Integrated Pinch Valves.
The invention also provides in one of its aspects, a microfluidic device in which the flow is controlled by valves able to open and close in a controlled and progressive manner, in order to avoid a disturbance of the array of capture objects, and a valve displaying such characteristics. A first exemplary way of providing this, is to use microfluidic channels with integrated valves, aimed at controlling the transport of fluids to and from the magnetic microcolumn array.
Such arrays can be prepared following a process inspired from xia and Whitesides, Angew. Chem, 1998, 110, 568-594. A double layer of positive photoresist (AZ9260) was spin-coated at 1000 rpm on a 2 in. glass substrate and patterned by standard photolithography. Once developed in Shipley 351 Developer, the photoresist was heated above its glass transition temperature at 150° C. during a few seconds, thus rounding the channel cross section. The channel has a width of 500 μm and a height of 50 μm at its highest point.

The master for the valve control layer was made by spin coating SU8-2075 negative photoresist onto a 2 in. glass plate and patterned following standard photolithographic protocols. The actuation channel has a rectangular section with 40 μm height and 250 μm width.

A 10:1 mixture of PDMS Sylgard 184 silicone elastomer and curing agent was poured onto the valve master to form a 5 mm thick layer. A 20:1 mixture of PDMS was spin-coated at 1600 rpm onto the master for microchannels for 30 s. Both layers were cured at 65° C. for 1 h. The valve layer was released from the mold. Valve actuation holes of 0.5 mm were punched. The valve layer was cleaned with isopropanol, dried with air and treated in an air plasma for 1 min to render its surface reactive. The valve layer was optically aligned to the fluidic channel layer. Bounding of the two layers was achieved for 2 h at 65° C. and the assembled layers were peeled from the fluidic channel master and fluidic 2.5 mm access holes were punched.

The PDMS fluidic channel complete with actuation channels were cleaned with isopropanol, aligned and sealed irreversibly on the coverslip bearing the ferrofluid pattern after 1 min exposure to air plasma.

In order to achieve a progressive control of these valves, which was not known in prior art, in this embodiment one can activate the control channels of the valves by a pressure controller, which is itself able to control pressures in a progressive and programmable manner, such as the MFCS from Fluigent. In a preferred embodiment, this flow controller may also be used to control, in a synchronized way, the pressure applied to reagents and or sample vials, in order to control their flow velocity in the device

### 7 External Progressive Pinch Valves

As an alternative to the above embodiment with valves integrated in the microfluidic chip, another embodiment, interesting in particular for flowing large quantities of liquids, it may also be useful to rather use progressive valves 80 not integrated in the microchannel array, such as represented in FIG. 8. This particular valve acts by pinching with a piston 81a flexible tubing 82 connected to an inlet 20 of the microfluidic chip. FIG. 8A represents a computer assisted design of the valve 80, and FIG. 8B a picture of the fabricated valve with its tubing in place. In contrast with pinch valves of prior art, in which the pinching is obtained in one shot, e.g. by a solenoidal piston, here the movement of the blade 83 pinching the tubing 82 is controlled by a motor, for instance a stepping motor 84, the speed of which can be controlled in order to impose the desired speed of closure and opening.

Such a motorized pinch valves 80 to control fluid flow in a microfluidic device, is particularly useful within exemplary embodiments of the invention, in order to control and automate flows within the microfluidic system of the invention, without the pulses that affect conventional valves such as ordinary pinch valves, “quake” microfabricated valves, or rotary valves. These new valves of the invention are designed to be mounted on the external tubes supplying any microfluidic system and may provide a versatile miniaturized system to pinch tubes with a large variety of sizes (from 1 to 5 mm). The global shape of the valve was optimized to provide easy of the valves on optical microscope stages and rapid installation on the tubes (removable end part). FIG. 8A shows 3D view of an exemplary design of valve.

This embodiment uses a DC-motor 84 10*24 mm from FAULHABER® (1024 M 012 S) equipped with a gear-head (10/1 256:1) to decrease the speed down to 1 round per minute. This should provide an accurate control over tube closing. FIG. 8B shows images of the pinch valve coupled to a PDMS microfluidic device through a 3 mm diameter silicon tube.

The monitoring and control of the electrical current value through the motor gives a direct access to the motor torque and thus to the forces applied on the tube during the closing and opening steps. This feature also provides the opportunity of using the valve as flow controller.

First experimental characterization of the valve performances showed perfect sealing for fluid pressures up to 2 bars (1 h experiment). Maximum opening and closing times may be tuned at any value above 2 s with the motor and gear-head described above, and shorter time constants may be achieved if wanted with motors with a lower gear ratio.

### 8/ Example of the Magnetic Activation of Activable Capture Objects of the Magnetic Type in the Invention

Once the microfluidic array has been assembled and connected to the different vials for reagents 11 and samples 9, in some embodiments, notably those in which the capture of analytes is indirect, it may be needed to assemble the capture objects onto the capture elements. This is done the following way:

a/ A suspension of capture colloidal objects (e.g. here magnetic beads) are flown in the active zone 23, by activating the control valves 80 and flow control elements as described e.g. in part 6/ or 7/,

b/ A magnetic field is applied through the active zone 23, said active zone 23 preferably bearing capture domains prepared according to parts 2/ or 3/. Means to activate such magnetic field are described in part 9/ below. Preferably, said field is essentially uniform on a scale larger than the typical distance between capture elements 3, and its amplitude can be controlled, in a continuous manner.

c/ flowing in said at least one active area a fluid sample containing analytes.

The method may comprise in addition a rinsing step d, performed between steps b and c above, in which said active area is rinsed with a fluid containing no capture colloidal objects able to assemble onto said capture elements 3, and no analytes.

The method may further contain additional steps e, following step c, in which reagents are flown into said active area whereas said first means are kept activated.

As an example, the magnetic particles are Dynabeads, 4.5 μm. However, depending on the application a large, variety of magnetic beads, with a large variety of sizes ranging from 20 nm to 20 μm, commercially available or prepared according to the art, may be used. For instance, one may use other smaller beads from DYNAL®, with diameters 1 μm or 2.8μ, or a variety of beads by competing companies like ESTAPOR®, ADEMTECH®, POLYSCIENCES®, IMMUNICON®, and others.

Beads with a larger magnetization and a larger size tend to yield stronger immobilization power, as will become more apparent in part 4/, so preferably, beads used within the invention should have a diameter of at least 200 nm, preferably at least 500 nm, yet preferably at least 1 μm, at least 2.5 μm, and some preferred embodiments, particularly suitable for high throughput cell sorting, at least 4 μm.

In exemplary embodiments, the invention involves flowing inside the active zone, while magnetic field is inactive, a suspension of magnetic beads with a size polydispersity at most 2, preferably at most 1.5, and even preferably at most 1.2.

As mentioned above, in the particular embodiment described here, the above magnetic beads carry on their surface ligands suitable for capturing the analytes of interest from the sample.

In some cases, however, it is not easy, or even impossible, to find or to prepare magnetic beads that have all the physical properties described above, and at the same time carry the right ligand for a given application. In that case, the two different populations of magnetic beads may be used. In other exemplary embodiments of the invention, in a first step a first population of magnetic particles with a first size, suitable for strong immobilization on magnetic domains, but not carrying ligands for the analytes of interest, are flown in the active zone in the absence of magnetic field, the flow is stopped, and in a second step, the magnetic field is then applied and the first population of magnetic particles are organized. Optionally, a rinsing step is applied, while keeping the magnetic field activated, and then in a following step, a second population of magnetic particles, carrying ligands to analytes of interest, and having a size or magnetization significantly smaller than that of said first magnetic particles, are flown in the active zone, and attach to said first population of magnetic particles by magnetic interaction. In other embodiments, said first and second populations of beads are flown in the active zone simultaneously.

More details about fluidic implementation are given below regarding some examples of application, notably for the typing of cancer cells.

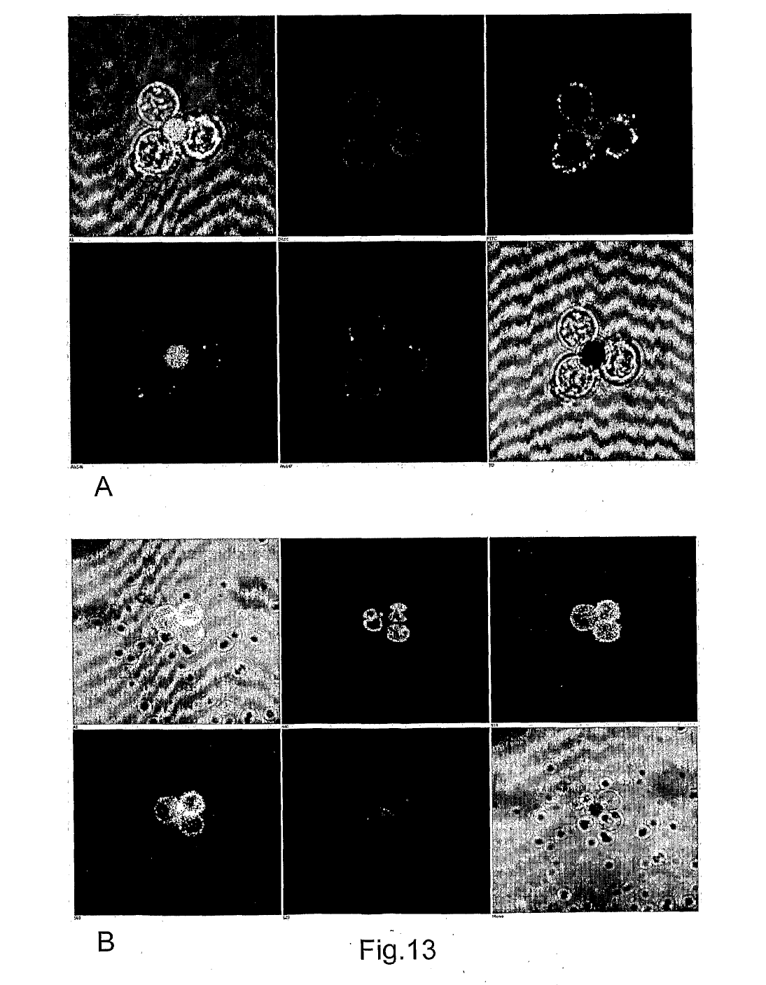

Alternately, one may capture analytes with the same ligand, but subsequently treat the at least two active zones, as appearing e.g. in FIG. 13, with different ligands, in order to reveal different biomarkers. Finally, one may also flow in at least a first capture zone a first sample, and in a second capture zone a second sample. This latter embodiment may be very useful, for instance for studying the effect of different stimuli on different cell populations, or for comparing different body fluids or different tissues from the same patient. All these various embodiments involving differential use of at least two active zones in the same chip, may be easily implemented by those skilled in the art, by minor variations with regards the layouts presented in FIG. 1, FIG. 5 or FIG. 6.

Finally, the invention may also enable the application of complex cell characterization and labeling protocols, in a highly automated and highly reproducible manner. This is demonstrated in examples of cancer cell typing below

Several advantages of the invention may stem from the possibility of flowing, in a controlled way samples and reagents, in arrays of colloidal objects, maintained in their position reversibly by an external field. If flow were too irregular, and notably involved the pulses associated with the brutal opening or closing of valves, as in prior art, said colloidal objects might be perturbed, and the advantages of the invention lost in some cases.

### 9 Exemplary Embodiments of Components Suitable for Activating and Deactivating Capture Elements.

A definite advantage of the invention, as compared to prior art, is the possibility to activate and deactivate capture elements or capture objects. We consider here as an example, capture elements that are magnetic. As shown in FIG. 1, capture elements 3 may be activated by applying a magnetic field thanks to an electromagnet or an electric coil 7. However, this may have some disadvantages, notably regarding electric consumption, and the need to cool the coil 7. It is thus one of the objects of the invention to provide ways of magnetically activating capture elements, that do not suffer from these disadvantages. Notably, in preferred embodiments, one needs a way to increase or decrease the amplitude of the magnetic field in the active zone, while keeping it essentially uniform all across the active zone.

A first embodiment, schematically represented in FIG. 9A, consists in “sandwiching” at least one microfluidic chip 1 comprising the active zone(s) between two parallel flat magnets 30 and 31, with North and South poles facing each other. Preferably, the smallest dimension of the magnets, in the plane of the chip, is larger than at least 3 times, preferably 5 times, the largest dimension of the chip 1. A translator 33 allows to increase the gap between the magnets 30 and 31 in order to reduce the field while keeping it essentially perpendicular to the chip 1 and uniform, in the central zone of the magnets. Optionally, at their wider spacing the magnets can be “docked” in a magnetic shunt 34, in order to reduce the magnetic field to about 0. Optionally, at least one of the magnets may comprise in addition one or several hole(s) 35 for tubings towards the chip 1.

This embodiment may also be particularly interesting for high throughput applications. In such applications, several microchannels arrays according to the invention may be stacked between the magnets, and operated in parallel. In combination with this embodiment, fluidic connections may be located on the side 37 of the chips, rather than on their top. In combination with this embodiment, too, it is preferable to use microfluidic devices of the invention with a low thickness, typically less than 2 mm, preferably less than 1 mm each. As a matter of example, said devices may have the format of a CD or a mini-CD.

A second embodiment, that may be preferred if one wants to observe the active zone(s) by an optical means in the presence of the field, is represented in FIG. 9 B. In that case, the chip is placed at the center 40 of a series of several magnets 41 with parallel polarizations in a circular arrangement, and the magnets are moved radially in order to increase or decrease the field. Optionally, in their most distant position, the magnets 41 can be docked in individual or collective magnetic shunts. FIGS. 10 A and 10B represent the computer assisted design of the magnets and their shunts in 3 D (upper left) and top view with magnetic field in false colours (upper right) and the COMSOL simulation of the field along a vertical central axis (lower left) and along a diameter (lower right) One can note in particular that for FIG. 10 A, the field is about 0.1 Tesla and reasonably uniform on one half of the distance between magnets, whereas in FIG. 10 B it is essentially zero.

10/ Examples of Methods that can be Useful for the Characterization and Optimization of Magnetic Particles Immobilization and Flow, within the Invention

The general operation of the system for immobilizing capture objects on capture elements was presented in FIG. 9. The magnetic beads, in suspension in water or buffer, for example additioned with a concentration below 0.01% and 1% of non-ionic surfactant, are introduced into the separation channel under microfluidic control.

Flow is then arrested, and the magnetic field is immediately applied. The beads self-organize in columns over the ferrofluid dots, as expected (FIG. 4C). We noticed that when a moderate flow, for example of the order of less than 20 μm/s, is applied to the array and the magnetic field is turned off, the columns remain irreversibly bounded and attached to, the magnetic dots. Field-mediated bead-bead adhesion is observed as a consequence of the interpenetration of polymer or protein layers at the surface of the beads, under the pressure induced by dipole-dipole interaction (Goubault et al., Langmuir, 2005, 21(11), pp 4773-4775). For the optimal concentrations of the beads suspension, columns may have a height equal to the channel's thickness, and are made of single aligned beads with only few defects. A few “free standing” columns nucleate between the dots, but they are easily removed by a gentle flow applied while keeping the magnetic field on.

For lower concentrations, columns are incomplete, or absent from some dots.

For concentrations above the optimal one, in contrast, the number of “free standing” columns increases, and columns assembled on the magnetic dots tend to loose their cylindrical shape, and adopt planar arrangements. Ultimately, labyrinth structures are obtained instead of hexagonal arrays.

It was observed that when magnetic columns are assembled under optimal conditions, they start to detach when the maximal flow velocity (achieved in the midplane of the channel) is around 400 μm/s, and for buffer velocities in the midplane of the channel between 800 μm/s and 1 mm/s, all columns are detached from magnetic dots without damaging the latter, and the microchannel may be washed out. This is a considerable improvement with regards to non-templated magnetic arrays, which are destabilized for fluid velocities typically around 10 μm/s.

Optimal values of the flow velocity for cell capture are around 100 μm/s, typically between 50 μm/s and 200 μm/s, so the stability of the columns leaves a comfortable margin of operation, even taking into account the fact that cell capture may increase significantly the viscous drag on a given column.

Theoretical guidelines to optimize flow and magnetic parameters for different geometrical parameters of the device are provided below.

The column of magnetic beads is principally submitted to two forces, a magnetic force that maintains the column on the dot of ferrofluid and a hydrodynamic force, due to the flow, that lead to pull it out. Experimentally, it was shown that there was a liquid speed threshold from which the columns detached themselves from the dots. The objective of this study is to evaluate these two forces and to verify the coherence with the experimental measure.

Estimation of the Hydrodynamic Force

When a solid is in relative movement in comparison with a fluid, the fluid applies on this object a force that may be resolved in a drag force T parallel to relative velocity of the fluid (V) and a lift P, perpendicular to V.

$$T = {C}_{T} \cdot \frac{μ \cdot S \cdot {V}^{2}}{2} and P = {C}_{P} \cdot \frac{μ \cdot S \cdot {V}^{2}}{2}$$

With C_{T} and C_{P} respectively the coefficients of drag and of lift, S the surface of the solid projected on the plan perpendicular to V and μ the density of the fluid. C_{T} depends on the geometry of the solid and C_{T} depends on the Reynolds number of the flow.

In the case of a sphere in a uniform velocity field and for weak Reynolds number, C_{T} follows the experimental law:

$${C}_{T} = \frac{24}{ e} = \frac{12 \cdot υ}{r \cdot V}$$

with S=π·r² the radius of the sphere and U kinematic viscosity of the fluid. The drag force on a sphere is then:

$$T = \frac{12 \cdot υ}{r \cdot V} \cdot μ \cdot \frac{π \cdot {r}^{2} \cdot {V}^{2}}{2} = 6 \cdot π \cdot η \cdot r \cdot V ( Stokes ' law )$$

Nevertheless, our experimental system differs a little from this model because the column of beads is not in a uniform velocity field. The fluid flow in the channel follows indeed a Poiseuille's law:

$$V ( z ) = V max \cdot ( 1 - \frac{{( z - L / 2 )}^{2}}{{( L / 2 )}^{2}} )$$

with Vmax, the maximum speed reached by the fluid in the channel and L, the height of the channel and z, the height within the channel. Furthermore, each bead of the column modifies the Poiseuille's flow but we will neglect this coupling afterward. In a second approximation, we will consider that the relative speed of the fluid on a bead is the speed in its mass centre. In these conditions, one may evaluate the hydrodynamic force by:

$$T = {\sum }_{i = beads} {T}_{i} = 6 \cdot π \cdot η \cdot r \cdot {\sum }_{i = beadcentre} V ( {z}_{i} )$$

Estimation of the magnetic force The magnetic beads are superparamagnetic and the plot is made of ferrofluid. Without an external magnetic field, these objects are not magnetic, but when they are under an external magnetic field, they become magnetic, that is, they will have their own magnetization and modify the nearby magnetic streamlines.

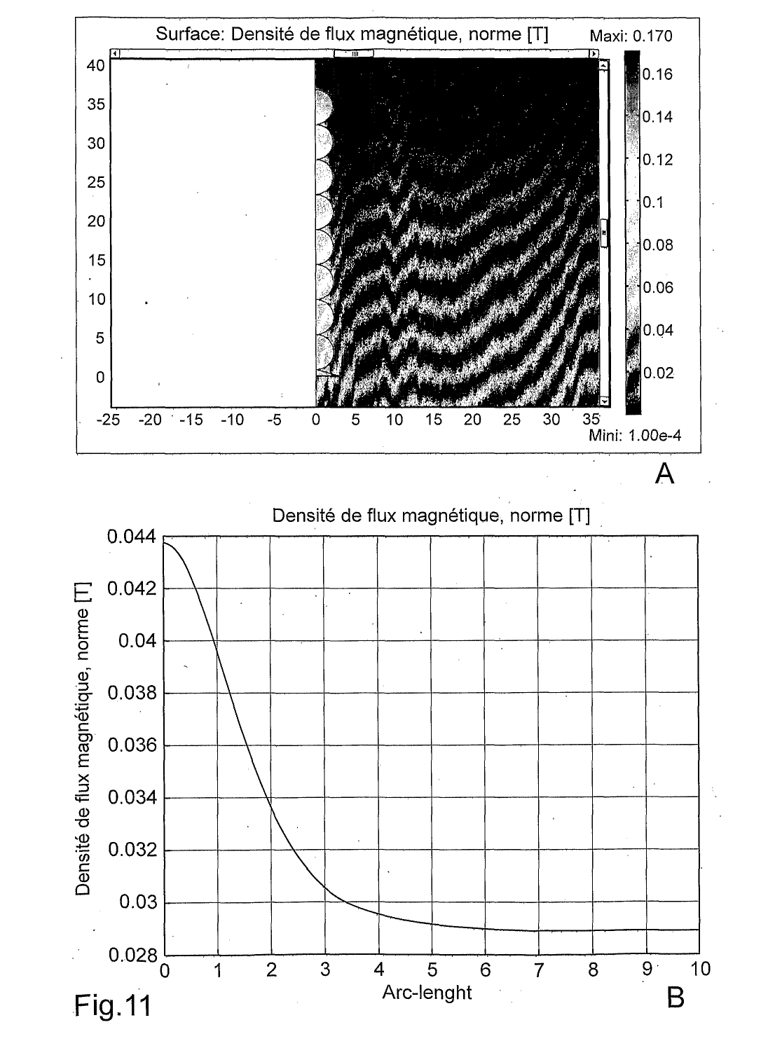

With COMSOL software, we have build a 2D axis y metric model, representing the system by a column of 8 spherical beads with a diameter of 4.5 μm, with a magnetic susceptibility of χ=2.6, located above a conical dot of ferrofluid with a basis of a height of 1 μm and a magnetic susceptibility of χ=3.3. FIG. 11A shows the intensity of the magnetic field of this model, for the case of magnetic capture elements prepared by microcontact stamping. The magnetic field being maximal on the tip of the cone, and its size being rather small, one will consider in a first approximation that the contribution of the plot is equivalent to a magnetic dipole, placed at the mass centre of the cone. Its magnetic moment is equal to the one of the cone. Besides, the magnetic contribution of the column being clearly superior to the one of the cone, we will consider that the magnetic disruption due to the cone on the field of the column is negligible. In these conditions, one may evaluate the force that keeps the column on the plot by:

$${{F}_{mag} = \vert {\overline{F}}_{plot \to column} \vert = \vert {\overline{F}}_{column \to plot} \vert = \overline{m} \cdot \overline{grad} ( {B}_{column} ) \approx m \cdot \frac{\partial {B}_{column}}{\partial y} )}_{max}$$

The magnetic field on a horizontal line 0.6 μm under the column (FIG. 11B), and the magnetization of the cone without the column have been measured by the software.

Those skilled in the art will be able to adapt this method of modelling to various kinds of capture elements.

Impact of the Parameters

The drag force on the column depends principally on Vmax and of L. For a define height of the channel, for example of 38 μm, the drag force varies linearly with Vmax: T=2.87 10̂(−7)·Vmax. For a maximum relative speed of the fluid of 1 mm/s, one obtains: T (1 mm/s)=2.38 10̂(−10) N. If the speed varies of 20%, one obtains T(1.2 mm/s)=2.86 10̂(−10) N and T(0.8 mm/s)=1.9 W(−10) N.

The magnetic force depends on the magnetic moment of the ferromagnetic cone and on the magnetic gradient due to the column. These two components vary with the intensity of the external field; the first one varies also with the volume of the cone and the second one with the height of the column.
Influence of the length of the magnetic column: the magnetic gradient due to the column varies quite little with its length. For an external field of 28.9 mT, the maximum of the magnetic gradient due to the column is of 6.8 10̂(−3) T/μm for a column to 8 beads and 6.69 T/μm for a column to 2 beads, the variation is then of 1.6%.
Influence of the external magnetic field: for a conical dot with a diameter of 5 μm and a height of 1 μm, and a column of 8 beads, the magnetic force is of 6.73 10̂(−10) N in an external field of 28.9 mT and of 5.1 10̂(−10) N in an external field of 25.1 mT, i.e. a variation of 24.3%.
Influence of the cone volume: the volume of the ferromagnetic dot influences in a major manner the magnetic forces. For a column of 8 beads, in an external magnetic field of 28.9 mT, magnetic force is of 9.43 10̂(−10) N for a dot with a diameter of 6 μm and a height of 1 μm, and of 4.49 10̂(−10) N for a dot with a diameter of 4 μm and a height of 1 μm, i.e. a variation of 52.4%.

Experimentally, we were able to verify that the ferrofluid dot has a diameter of about 5 μm and a height of 1 μm, and that the external field is about 28 mT. The magnetic force for these values is estimated at 6.7 10̂(−10) N.

The results obtained with these models are therefore consistent, for the two forces in competitions are well in the same magnitude order, showing that the model is indeed usable for predicting the magnetic resistance of the columns, and preparing, with the description above, numerous other variant embodiments of the invention.

### 11/ Evaluation of Cell Capture Efficiency on Cell Lines

### Cell Lines Culture and Preparation

Cell culture reagents were purchased from Invitrogen. B lymphocytes “Raji” (ATCC CCL-86) human cell lines were cultured in RPMI 1640 supplemented with 100 U/mL aqueous penicillin, 100 μg/mL streptomycin and 10% fetal bovine serum. Epithelial cells “MCF7” were cultured in DMEM supplemented with 100 U/mL aqueous penicillin, 100 μg/mL streptomycin and 10% fetal bovine serum. Cells are cultured at 37° C. in a humidified atmosphere with 5% CO₂.
In experiments with spiked cell lines, long term cell tracker dye, green 5-chromethylfluorescein diacetate (CMFDA) (Invitrogen, France) was used to distinguish one population from the other: MCF7 cells were incubated in phosphate buffered solution 1× (PBS, pH7.4, Gibco, France) containing 1 μM of CMFDA for 30 min at 37° C. Cells were then washed and incubated in culture medium for 30 min at 37° C. Cells were then washed and resuspended in PBS solution supplemented with 0.1% bovine serum albumine (BSA) obtained from Sigma (France. Concentration of cell suspensions was measured using a hemacytometer.

### Quantification of Cell Separation

In order to quantify separation yield and selectivity, an array of anti-EpCAM labelled magnetic beads were formed: beads were injected in the channel, flows were stopped, the magnetic field was applied, beads in excess were washed with PBS. MCF7 cells were labelled with CMFDA CellTracker as described above. A mixture of MCF7 and Raji cells was prepared and cell number of each population was quantified with a Malassez chamber.
If using blood, MCF7 were added to 0.5 mL of blood.
If using FACS lysing buffer, 0.5 mL of cells was incubated in 5 mL of lysing buffer 1× for 15 min at room temperature. Then cells were centrifuged for 10 min at 400 g and supernatant was discarded and only 0.5 mL of cell suspension was kept.
If cell sample has only to be analyzed by fluorescent labelling (blood sample or lumbar puncture from cancer patient), there is no need of using CMFDA labelling.
The mixture was immediately loaded in the microfluidic chip, to avoid adhesion between these two cell populations. The microfluidic channels were then washed with PBS+0.1% BSA, to remove uncaptured cells. Fluorescent cells were counted to determine capture yield and capture profile across the magnetic array.

### Evaluation of the Capture Performance of the Invention Regarding Cell

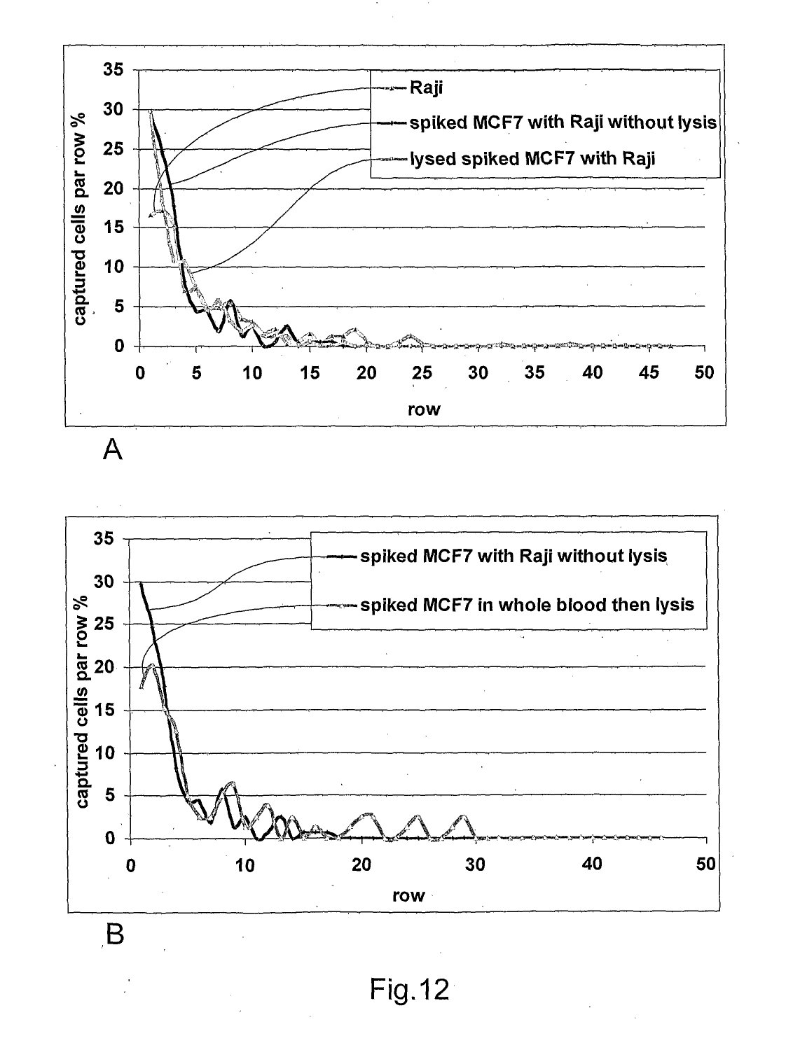

The efficiency of the invention for capturing cells was evaluated Cells are captured in a microfluidic device, with a layout corresponding to this described in FIGS. 6C and 6D, onto an array of magnetic beads coated with specific capture antibodies, and assembled as described in part 8/. The array of capture elements was prepared according to part 3/, and the microfluidic chip prepared according to part 5/.
In a first series of experiments, some lymphocytes (Raji cell line) have been introduced in the device and captured by anti-CD19 Dynal beads. Number of cells captured per row of beads was measured.
Some epithelial cancer cells (cell line MCF7), expressing the surface antigen EpCAM, were stained with CMFDA (CellTracker™ Green CMFDA, Invitrogen). A known number of MCF7 were spiked in a buffer containing lymphocytes (Raji cell line) at a ratio 1 MCF7/10000 Raji. They were captured by anti-EpCAM Dynal beads. Global capture yield was 75+/−10%. Number of cells captured per row of beads was measured as a function of the penetration depth of the cells in the array before their capture (FIG. 12A). This shows that the capture efficiency of the invention is extremely high, and that most of the cells are captured, in this particular embodiment, before row number 15.
Then, one studied the efficiency of the system to capture cells from a blood sample in which red blood cells have been lysed Blood sample are very viscous, so in order to introduce raw blood in the array without damaging the magnetic array of columns it would be necessary, within the invention, to use a low flow rate. Thus, in a preferred embodiment, red blood cells are selectively lysed in order to decrease the number of cells (and consequently, the viscosity) in a blood sample. Using flow cytometry we checked that the EpCAM and CD45 antigens were not damaged following a lysing step with FACS lysing buffer (BD Bioscience).
Finally, MCF7 cells were spiked at known concentrations in 0.5 ml human blood. Red blood cells were lysed (FACS lysing buffer, BD Bioscience). Nucleated cells were resuspended in 0.5 mL of PBS. A capture yield of 60+/−5% was obtained, and number of cells captured per row of beads was measured (FIG. 12B).
Also, it is seen that the efficiency of capture per row remains high, since all cells are capture between row number 1 and row number 30. This is a considerable advantage as compared to prior art, notably Nagrath Nature 2007 nature Vol 450|20/27 Dec. 2007 In this device of prior art the active domain is an elongated parallelepipic volume, with a high footpring, for example 19 mm width for 51 mm length, with an inlet and an outlet. The cells are captured on permanent obstacles of large size, as shown by scanning electron microscopy In the invention, a same throughput can be achieved in a system with a much smaller footpring, notably thanks to a much shorter length of the capture zone, here 3 mm This is also an advantage with regards to the layout disclosed in Saliba et al., Proceedings uTAS 2007 (The 11th International Conference on Miniaturized Systems for Chemistry and Life Sciences), Paris 2007, ISBN 978-0-9798064-0-7, Publisher CBMS, Cat Nb 07CBMS-0001, in which the active zone has a length larger than its width, and thus cannot produce a high throughput.

### 12/ Labelling Protocol for High Resolution Cancer Cells Characterization Antibody Fluorescent Labeling Protocol

In order to allow for high resolution microscopy observation, specific fluorescenty labeled antibodies had to be prepared. Cell labelling of specific protein (Cytokeratine, CD45) is realized by conjugating a specific antibody with fluorescent anti-IgG antibody provided by Zenon Mouse IgG Labeling Units (Invitrogen):
1 μg of antibody is diluted in phosphate-buffered saline (PBS) (=20 μL. 5 μL of the Zenon mouse IgG labeling reagent (Component A) is added to the antibody solution and incubated for 5 minutes at room temperature. 5 μL of the Zenon blocking reagent (Component B) is added to the reaction mixture and incubated for 5 minutes at room temperature. The complexes are then ready and should be applied to samples within approximately 30 minutes.

### Immunofluorescent Cell Characterization

After cell capture in the magnetic array, cells nucleus were stained with Hoechst 33342 (Invitrogen, France) by incubating cells for 30 min at room temperature. After a washing step with PBS, cells were incubated for 30 min with antibody labelling mixture—anti-CD45 conjugated with Alexa Fluor 488 with Zenon unit. Microfluidic channels were then rinsed with PBS and cells were subsequently fixed in the chip by a 3,7% paraformaldehyde (Sigma) solution during 15 min, and rinsed with PBS for 15 minutes. Then their membranes were permeabilized by incubating the sample in PBS containing 0.1% TritonR X-100 for 5 minutes at room temperature and rinsed with PBS for 15 minutes. Cells were then incubated for 30 min with antibody labelling mixture—anti-EpCAM conjugated with Alexa Fluor 555 with Zenon unit. Finally, cells were fixed in the chip by a 3,7% paraformaldehyde (Sigma) solution during 15 min, and rinsed with PBS for 15 minutes. In order to perform more conveniently ex-situ analysis through laser confocal microscopy, the magnetic columns and cells were finally stabilized by flowing in the chip a 1% solution of low-melt agarose (Euromedex) in PBS. Agarose is gellified by decreasing the temperature in chip. The temperature in chip can be controlled by monitoring the temperature of the magnetic coil surrounding the chip (another systems with peltier device may also be used).
Following this procedure, the array of beads columns and the cells attached to them are maintained in position even after switching off the magnetic field, and the microfluidic chip can be disconnected from its fluidic control system and transported for imaging. Occasionally, during manipulation, the whole array of magnetic columns may be shifted with regards to the magnetic template, without impeding subsequent imaging.
13 Use of the Invention for the Typing of Leukemia from Patient Blood On-Chip Cell B-Cell Malignancies Immunophenotyping

Venous blood was sampled from B-cell malignancies already diagnosed in patients, within a personal data anonymization protocol, from Institute Curie Hospital, into EDTA tubes and was processed immediately. Leucocytes were isolated from red blood by Ficoll (Lymphoprep Cells nucleus isolated leukocytes was stained with Hoescht nuclear dye (Invitrogen, France) following instructions.

A microfluidic chip is prepared according to examples above, of the type with integrated microvalves. It involves PDMS microfluidic channels, with a thickness of 50 μm in the active area bound onto glass microscope coverslip with a 42 mm diameter and a thickness 170 μm. The coverslip bears on its surface magnetic domains as indirect capture elements, prepared according to part 1/. The chip is connected to a MFCS 8C (Fluigent) for automated infusion of samples, and anti-CD19 magnetic beads (DYNAL®1) are flown into the array, according to part 4/. A magnetic field of 25 mTesla, essentially uniform and perpendicular to the chip surface, is applied by activating a magnetic coil surrounding the chip.

Then 10 μL of leucocyte solution are introduced in the chip. After elution of about 8 μl, B-Cell captured are washed with PBS-1% BSA and Fetal Bovine Serum to block unspecific antibody capture sites.

A mixture of fluorescently labelled antibodies is then sent in the chip. The antibodies are in this particular example mAb anti-CD5 Alexa Fluor 488 labeled (BIOEGEND®, France), mAb anti-CD10 Alexa Fluor 555 (BD BIOSCIENCES®, France conjugated with Zenon Unit, Invitrogen) and a mAb anti-CD23 Alexa Fluor 647 (BIOLEGEND®, France). After 30 minutes incubations, cells are rinsed with PBS-BSA 0.2%. Cells are subsequently fixed in the chip 30 minutes with 3,7% paraformaldehyde (SIGMA®, France) and rinsed with PBS 30 minutes. In order to be analysed by laser confocal microscopy, cells are embedded in agarore gel (Low melt, SIGMA®, France) 1% in PBS to maintain the 3D structure of the array. Cells are analysed with NIKON® A1R confocal microscope.

Results

Patient 1: Chronic Lymphocytic Leukaemia

FIG. 13A displays a panel showing 3 B cells captured from a Chronic lymphocytic leukemia patient. Images correspond to a selected cut from 3D confocal stacks, recorded in different light channels corresponding to the different dyes involved, and from bright field transmission images.

In said panel,
upper left is a merge image,
upper middle is a Hoescht staining,
Upper right is CD23 immunolabeling,
Lower left is CD10 labeling,
Lower middle is CD5 labeling and,
Lower right is a bright field image.

For presenting the images, confocal stacks of images were selected to remove stay light from different sources, in particular fluorophores adsorbed on microchannel's surface, leading to much better signal to noise ratio. The peripheral colouring in panels C and E, provide clear evidence that the localization of the antigen is a membrane localization.

One may note that the magnetic beads used as capture colloidal objects have an autofluorescence in the green (upper right) and yellow (bottom left) channels, so that they can be used as a reference signal to obtain, regarding the analytes, a more accurate quantitative signal.

Patient 2: Acute Lymphoblastic Leukaemia

FIG. 13B shows images from cells from a patient subject to Acute Lymphoblastic Leukemia, obtained in conditions similar to those used for FIG. 13A. The phenotype is this time CD23+, CD10+, CD5−. This result is in accordance with in cytometric data obtained in parallel.

14/: Use of the Invention for the Sorting of Rare Cells from Large Sample Volumes

To quantify the performance of the sorting device, B-lymphocytes expressing CD19 membrane protein (Raji cell line, CD19+) mixed with T-lymphocytes (Jurkat cell lines, CD19−) were detected. Target cells are in this example recognized using a green cell tracker CMFDA dye. The capture of epithelial cells (MCF7 cell line) spiked in a mixture of lymphocytes was also studied, as a model for the dissemination of epithelial cells from a primary tumour (e.g. breast cancer) in blood. Experiments were also realized with whole blood sample.

Hexagonal arrays of ferrofluids spots are formed by micro contact printing on a cover glass. PDMS microchannels are sealed after a plasma treatment. The flows of reagents are controlled dynamically using a MFCS. Magnetic beads (Ø=4.5 μm, DYANL®) are injected in the separation channel and self-organize into columns over the ferrofluid spots, using a 30 mT magnetic field. The beads are grafted either with anti-CD19 antibody or anti-EpCAM antibody, depending on the targeted population of cells.

After rinsing, the cell mixture is injected in the sorting system. Finally, rinsing solutions and reagents for staining are sequentially injected under MFCS fluidic automation. Raji were stained in-chip with anti-CD19-AlexaFluor488 (membrane), Hoechst (nucleus) and with May-Grunwald-Giemsa reagents after fixation (for assay of cell-morphology). MCF7 captured cells are stained with anti-EpCAM-AlexaFluor488, Hoechst, and anti-cytokeratin-AlexaFluor594 after fixation. Cells are observed in bright field and in fluorescent light at high magnification.

Cell mixture with 1 positive cell per 1000 negative are studied. The global capture yield is 55+/−10%. the cell velocity during the capture is about 300 μm/s and the throughput is 0.5 mL/h with the geometry of FIG. 5A

With the geometry of FIG. 6A), we expect a throughput of about 3 mL/h with cell lines mixture with a total cell concentration of about 10̂6 cells/mL. Experiment conducted with whole blood sample diluted twice in PBS showed that columns don't resist at a flow velocity exceeding 0.3 mL/h, because of the higher viscosity of whole blood comparing with sample containing cells at 10′6 cells/mL. Thus, when flow throughput larger than this value, and with a total section as that presented in this particular example, 33 mm×0.05 mm×2=3.3 mm² (number of active zones, multiplied by width of the active zone, multiplied by thickness of the active zone, the invention is preferable associated with a step of red blood cell lysis.

Alternately, if one wants to avoid RBC lysis, one may increase the total sectional area of active zones, e.g. by increasing their width, or the number of active zones.

Cell-line culture. The human breast cancer cell line MCF7 is maintained and grown to confluence in DMEM (Invitrogen) medium containing 4 mM L-glutamine supplemented with 10% fetal bovine serum and 1% penicillin-streptomycin liquid (Gibco) and lymphocytes cell lines (Raji and Jurkat) are grown in RPMI-1640 with GlutaMAX (Invitrogen) supplemented with 10% fetal bovine serum and 1% penicillin-streptomycin liquid (Gibco) both at 37° C. in 5% CO2, with humidity.

To dissociate MCF7, medium is aspirated and cells are resuspended, centrifuged 5 min at 300 g and rimed twice with HBSS and incubated with trypsin-EDTA for 2 min. Cells are then resuspended and rinsed in PBS+2 mM EDTA+0,1% BSA. Lymphocyte cells are resuspended, centrifuged 5 min at 300 g and rinsed in PBS+0.1% BSA at a concentration of 10^{7′6} cells/mL.

Spiking experiment. Cells to be spiked (MCF7 or Raji) are pre-labelled with CMFDA cell tracker using the standard protocol provided by the manufacturer. The cell titre is determined by counting with a haemocytometer. The desired concentration of cells is then prepared by serial dilution of the original cell suspension in PBS+2 mM EDTA+0.1% BSA. Labelled cells are spiked at a concentration of 1/1000 with Jurkat lymphocytes (which are at a concentration of about 10̂6 cells/mL) and injected in the sorting system. Captured cells are counted are discriminated by fluorescence.

Fixation and staining. Captured cells are fixed by incubating them with PBS+4% formaldehyde during 15 min and then washed with PBS during 15 min. cells are then incubated with PBS+0,1% Triton. X-100 for 5 min and washed with PBS during 5 min, Captured MCF7 cells are stained with incubation during 30 min with anti-EpCAM-AlexaFluor488 and anti-eytokeratin-AlexaFluor594. anti-EpCAM-AlexaFluor488 and anti-cytokeratin-AlexaFluor594 are obtained by mixing anti-EpCAM-IgG and anti-cytokeratin-IgG with anti-IgG-AlexaFluor antibodies supplied by Zenon (Invitrogen) following the standard protocol provided by the manufacturer.

A staining of the nucleus of captured cells may also be done by incubating them with DAPI during 15 min. Cells are then washed with PBS. Visualization was performed following the same procedures as in part 5/.

Example of Cell Imaging with Confocal Microscopy

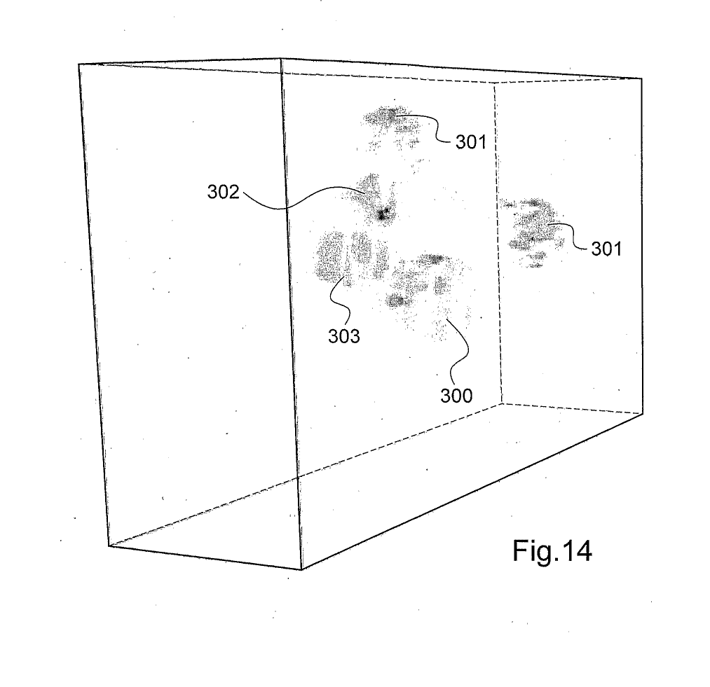

Cells were imaged by confocal microscopy, in the microfluidic system of the invention. An exemplary image is presented in FIG. 14

The colour labeling of antibodies is in this case is yellow for nucleus 300, respectively red, blue and green for surface antigens CD5 301, CD10 302 and CD23 303 (the image is presented in inverted colors, to avoid photocopy problems with color images on a black background. The three largest, cells on the right have a phenotype, CD5+, CD10−, CD23−, and the smaller one on the left, is CD5−CD10−, CD23+.

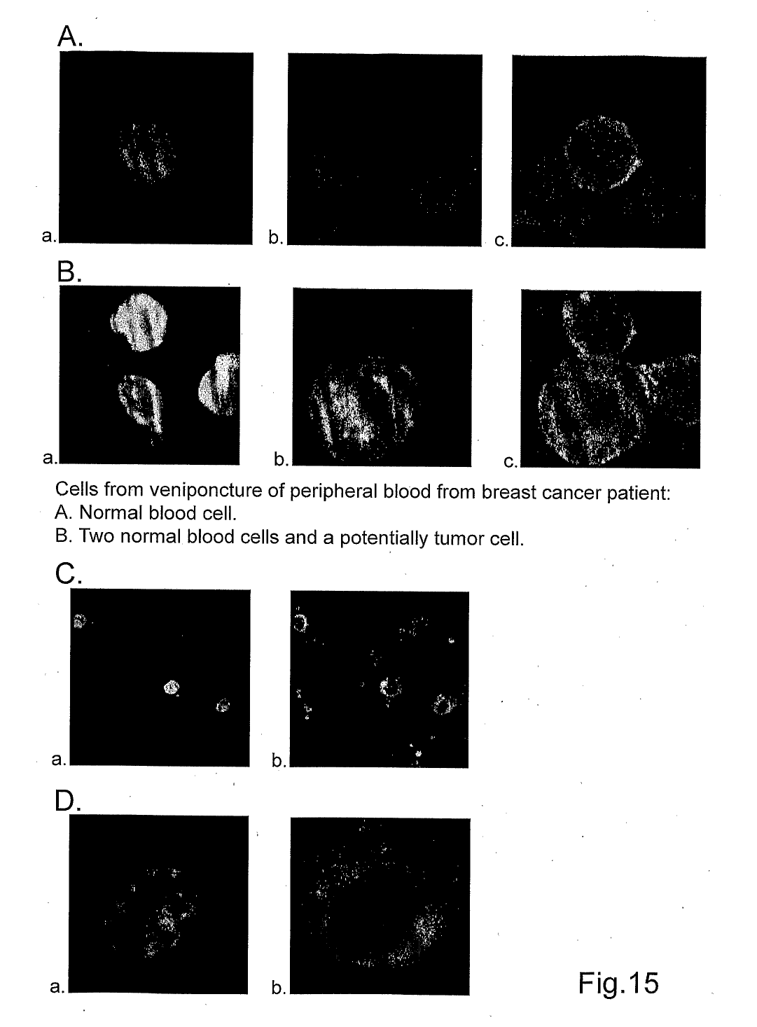

15 Immunophenotyping of Tumour Cells from Breast Cancer Patient.
Blood from patient was collected on a Veridex tube, and prepared the same way as in part 11/, except that in that case there is no need of using CMFDA labelling.
Red blood cells were lyzed with FACS lysing (BD Bioscience) following manufacturer protocols: buffer, 0.5 mL of cells was incubated in 5 mL of lysing buffer 1× for 15 min at room temperature. Then cells were centrifuged for 10 min at 400 g and supernatant was discarded and only 0.5 mL of cell suspension was kept.
The mixture was immediately loaded in the microfluidic chip, to avoid adhesion between these two cell populations. The microfluidic channels were then washed with PBS+0.1% BSA, to remove uncaptured cells. Fluorescent cells were counted to determine capture yield and capture profile across the magnetic array.
FIG. 15 represents images from cells from veniponcture of peripheral blood from breast cancer patient, obtained in a device according to FIG. 6D. FIG. 15A represents a normal blood cell and FIG. 15B represents two normal blood cells and a potentially tumor cell. FIG. 15Aa corresponds to the blue channel (hoescht staining), FIG. 15Ab. to the red channel (cytokeratine staining), FIG. 15Ac. to the green channel (CD45 staining).

### 16/ Improvement of Image Quality and Speed of Acquisition Using a Denoising Software and Spinning Disk Imaging.

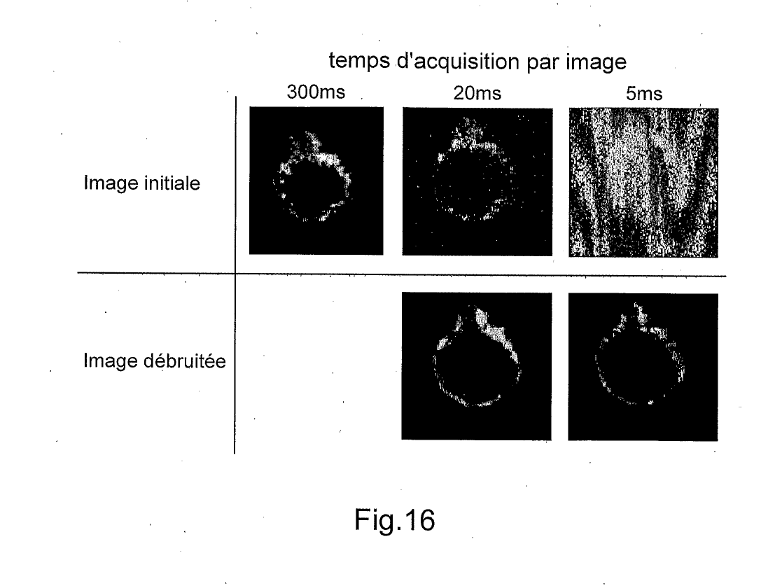

FIG. 16 represents images of a B lymphocyte (CD45 labelling) using a Yokogawa spinning disk system, Nikon inverted microscope, in combination with the invention: very fast acquisition of a complete cell image can be achieved while keeping a good quality of image, in 20 ins. In addition, a denoising software increases further the speed, allowing in this situation an acquisition time of 5 ms. The denoising algorithm may also be used with conventional imaging microscopy in combination with the invention, allowing to take advantage of its very high resolution imaging without compromising total acquisition time.

### ADDITIONAL ADVANTAGES OF THE INVENTION

As compared with conventional micro/nanofabrication tools, the use of indirect capture of analytes, which is enabled by exemplary embodiments of the invention, is low-cost, robust, and it permits high aspect ratio structures. It is reversible, so that a contaminated array may be replaced in a fully automated manner, as required for the development of an industrial instrument usable in routine practice. It may capture cells with yield and specificity better than 95%, and we could culture and reproduce cells on long time durations.

Exemplary embodiments of the invention also allow to fully automate complex protocols, such as cell staining, bioassay, DNA assay, protein assays, transcriptome analysis, genome analysis, directly in the chip in which the cells are captured, and thus considerably simplifies operation as compared to state of the art.

Besides the advantages recited above, exemplary embodiments of the invention offer the very unique possibility of applying to the captured cells, essentially all the most sophisticated tools under development for cell biology, such as microfluidic environment control, and high resolution automated optical screening.

Indeed, exemplary embodiments of the invention combine in a single method and device, the respective advantages of different state of the art techniques, and add some of its own.

Regarding visual cytometry, exemplary embodiments of the invention allow the application of all the staining and observation protocols currently used in optical cytometry. Because the cells of interest are sorted from the vast amount of unwanted cells, however, the total surface to screen is considerably reduced so that the screening may be performed at higher resolution in a shorter time, without overlapping with white blood cells and with considerably less human involvement. Finally, exemplary embodiments of the invention allow to fully automate the staining and observation steps, without the need for expensive robots, and without manipulation between slide preparation robots and the microscopy observation platform.

With flow cytometry, the invention shares the advantages of simplicity of use, high automation and quantification.

As flow cytometry, it offers the possibility of simultaneous, quantitative, multicolour typing of cancer cells, with regards to a multiplicity of fluorescently labelled biomarkers, along complex protocols. It presents, however, major qualitative improvements. First, it allows a considerable extension of the range of sample volumes amenable to analysis, in both directions: Thanks to its highly integrated and microfluidic nature, for samples containing a large fraction of cells of interest, it will yield statistically valid full typing with volumes typically in the range 10-20 μl, 10 times less than with flow cytometry. The invention, however, will also be able to process several ml per hour, and thus sort CTC from blood, an application out of reach of flow cytometry.

It is thus another object of the invention to provide a method for the sorting or the analysis or a combination of sorting and analysis, of analytes and notably cells, combining the advantages of flow cytometry and of visual cytometry.

More specifically, the invention provides a method for the sorting and or the analysis or a combination of sorting and analysis, of analytes and notably cells, comprising the steps of
capturing said analytes with magnetic particles
imaging or analyzing said analytes
extracting from said image or from the data resulting from said analysis at least one quantitative numerical result regarding at least one predefined criterion, and preferably more than 3, more than 5, more than 7, and up to more than 9 predefined criteria, said extraction being performed for at least one analyte, preferably for several analytes, more preferably for as many analytes, and ideally for all analytes captured
comparing said quantitative numerical results with a reference value

Optionally, the invention may usefully comprise steps consisting in plotting the data obtained above in multidimensional maps, as performed e.g. in flow cytometry.

Optionally, too, said reference value can be obtained either by the analysis of isotypes in the same conditions as used for analysis of the analytes, or by use of an internal reference. Said internal reference may, for instance, be provided by the signal emitted by some cells naturally contained in the sample, such as haematopoietic cells, or by pre-labelled cells spiked in the sample prior to analysis, or by labeled colloidal objects introduced in the sample prior to analysis, or to signal-emitting components introduced in the active zone during microfabrication, or by labeled colloidal objects introduced in at least one reagent flown in the active zone before or after the capture or analytes; Said reference may also be provided by some intrinsic property of the capture colloidal objects.

With regards to filtration on calibrated membranes, exemplary embodiments of the invention present the major advantage of allowing a selection of the cells of interest by a specific biomarker (or preferably by a combination of biomarkers, which may be either protein-based, nucleic-acid-based, or morphological). In addition, within exemplary embodiments of the invention the cells captured are presented on a much smaller area, in a format compatible with the most powerful micro and nano imaging tools, and with cell culture and in vivo studies.

Similarly to conventional magnetic sorting methods and devices, exemplary embodiments of the invention may use a large wealth of well characterized and highly specific functionalized magnetic particles in order to capture cells. It allows batch preparation, for a better routine quality control and run-to-run reproducibility, an essential property for passing certification tests and anticipating further standardization. This also considerably reduces production cost. Beyond this, however, the paradigm of magnetic sorting are completely reversed, bringing in a dramatic increase in sorting efficiency and sensitivity. Within exemplary embodiments of the invention, the magnetic particles are first immobilized, and then the cells are flown through. This presents two major advantages: first, the number of particles necessary is proportional essentially to the number of cells to be captured, and not to the total volume of the sample, as in state of the art methods.

Thus, for CTC, the total number of magnetic particles is reduced by orders of magnitude. This is interesting not only for cost, but also because the use of a large excess of particles leads to cell damage, reduces yield, and renders accurate morphological observation impossible. Second, in our system the interaction between the cells and the immobilized particles is not induced by Brownian motion, but by the well controlled hydrodynamic forces inside the array, resulting in considerably increase capture yield. Finally, exemplary embodiments of the invention also bring in considerable advantages as compared to previous microfluidic cell sorting methods, as described in the background above:

First, using a “bottom-up” activable self-assembly process instead of a “top-down” micro fabrication one in order to make our array of columns, within exemplary embodiments of the invention one may achieve higher aspect ratios, reduce considerably fabrication cost, and improve reproducibility. In addition, the reversible nature of the array allows for easy automation and industrialization, since contaminated arrays may be replaced by purely fluidic means.

The second major change is the possibility of performing highly automated high resolution microscopy and cell biology on the live captured cells. In previous art, cells are captured in microchannels on a thick microfabricated oxidized silicon wafer, so that the imaging tools that may be applied to their observation are limited, and low-resolution. With exemplary embodiments of the invention, in contrast, it will be possible to use conventional and unconventional microscopy methods. This advantage indeed makes the approach of the invention unique with regards to all methods existing or under development for the characterization of rare tumor cells.

As another advantage as compared to prior art, the invention considerably simplifies and accelerates cell screening operations, because it may perform sorting, capture, and analysis, in the same device. In microfluidic prior art, as disclosed e.g. in US2008090239, or US2007059680, there is a need for a first module for separation and a second module for capture, and even in some cases, a third container in which nucleic acids will be collected for further analysis. In prior art magnetic sorting devices, such as the one proposed by VERIDEX®, process also involves a lot o manipulation, and the use of two machines, one for magnetic sorting, and a second one for performing the analysis of the results.

It will be apparent to those skilled in bioassays, cell screening, cell-based diagnosis and prognosis, cell biology, developmental biology, drug discovery, drug screening, stem cells research, bacteriology, infectiology, biotechnology, that exemplary embodiments of the invention may have in all these fields important and useful applications.

Samples containing the analytes within the invention are for example fluid, but they may come from any origin. Particularly suitable are body fluids, such as blood, urine, plasma, serum, cerebrospinal fluid, lymp, saliva.

These samples may be used with some pre-treatment, such as centrifugation, ficoll gradient, selected lysis of some cells, precipitation, protein digestion, and any other treatment of interest for the particular application to which devices and methods according to the invention are applied.

Blood, for instance, may be used raw, preferably after collection on tubes suitable to preserve cells alive and avoid coagulation such as those sold by VERIDEX®, or other blood collection tubes. It may also involve, before use within the invention, a step of red blood cell (RBC) lysis, or a step of dilution. It may also, for some applications, involve a ficoll treatment, although in general one of the advantages of the invention is to avoid such ficoll treatment.

Samples may also provide from bone marrow, from biopsies, such as surgical biopsies, or aspirates, and notably fine needle aspirates (FNA). If sample is initially a solid one, it will preferably be treated prior to use within the invention, by a treatment suitable to dissociate and suspend cells in a liquid medium.

For environmental and security applications, samples within the invention may also come from the environment, i.e. crops, food, surface or subterranean water, sewage water, air.

Notably, a large number of potential applications of cell sorting and typing were recited in prior art, and notably in WO 2006/108087, US 2007/099207, WO 2006/108101, US 2007/196820, US2007/026-413, -469, -414, -415, -416, -417; -418; US 2007/059-716, -680, -774, -719, -718, -781; US 2007/172903; US 2007/231851; US 2007/259424; US 2007/264675; WO 2007/106598; WO 2007/147018; US 2008/090239; WO 2007/147079; WO 2008/014516; US 2008/113358, US 2008/0138809. We discovered that, surprisingly, these applications could indeed be addressed with the present invention more efficiently than with prior art devices.

Also, it should be noted that thanks to its very high efficiency of capture, the invention is able to capture without loss rare cells in blood, even after a step of lysis of red blood cells, and a step of reconcentration of the nucleated cells. This allows to significantly reduce the volume of the sample to be processed, and thus to increase throughput, as compared to prior art in which non-lysed blood had to be used.

Thus, it is also an objective of the invention, to provide a method for the capture, culture or sorting of analytes, and notably of rare cells, said method comprising a first step of providing a blood sample of volume A, a second step of lysing red blood cells from said sample, a third step of resuspending nucleated cells from said sample in a volume B, and a 4^{th} step of sorting a subset of nucleated cells from said volume B in a microfluidic device, wherein said volume B is less than 3 times, preferably less than 5 times, yet preferably less than 10 times, 20 times, 50 times or even 100 times smaller than said volume A.

Also the invention allows to collect cells from a large volume of initial sample into a small volume. As a matter of example, the volume of active zones in the embodiment described in FIG. 6, with a thickness of 50 μM, is about 5 μl. It is thus also an objective of the invention to provide a method for capturing rare cells from initial sample volumes of at least 1 mL, and preferably at least 5, 10, 20 and up to 50 mL, with an efficiency of capture of at least 20, preferably at least 40, 50, 60 and up to more than 80%, said captured cells being contained in an active zone of less than 50 μL, preferably less than 20 μL, 10 μL, and in some cases less than 5 μL.

The embodiments of the invention such as those presented in FIG. 6 may sustain flow rates of typically 1 to 3 mL/hour

Thus, with the volume reduction recited above, the invention may allow to sort rare cells, in less than 1 hour, with a ratio between the initial sample volume to the volume of the chamber in which said cells are captured larger than 100, preferably larger than 500, 1000, 2000, 5000, 10 000, and in particularly optimized cases even up to 100 000. Thus, it is also an object of the invention to provide a method for the capture of rare cells, comprising a first step of providing a first blood sample of volume A, and at least a second step of flowing said sample or a pretreated sample obtained from said first blood sample in an active zone or a combination of active zones, where said rare cells are captured, wherein said flowing step lasts less than two hours, preferably less than 1 hour, and even more preferably less than ½ hour, and wherein in less than 1 hour, with a ratio between the initial sample volume A to the volume of the active zone or the combined volume of the active zones in which said cells are captured is larger than 100, preferably larger than 500, 1000, 2000, 5000, 10 000, and in particularly optimized cases even up to 100 000

Associated with these advantages, the invention also provides a very strong reduction of the consumables, associated with the reduction of capture zone. For instance, the total mass of capture objects in an embodiment as presented in FIG. 6, is of the order of 50 μg. This is typically 100 to 1000 times smaller than the mass of beads used to analyse the same volume of sample, in prior art devices such as the Veridex system. Thus, it is another object of the invention, to provide a A method for magnetic capture of cells or analytes from an initial raw sample, such as for instance and non limitatively blood, wherein the total mass of magnetic particles used for treating least 1 mL, and preferably at least 5, 10, 20 and up to 50 mL of raw sample is less than 10 mg, preferably less than 5 mg, 2 mg, 1 mg, 0.5 mg, 0.2 mg, or less than 100 μg

Preferably, said capture occurs with an efficiency of at least 20, preferably at least 40, 50, 60 and up to more than 80%.

All publications and patent applications mentioned in this specification are herein incorporated by reference to the same extent as if each individual publication or patent application was specifically and individually indicated to be incorporated by reference.

The indefinite articles “a” and “an,” as used herein in the specification and in the claims, unless clearly indicated to the contrary, should be understood to mean “at least one”.

As used herein, “or” should be understood to mean inclusively or, i.e., the inclusion of at least one, but also possibly more than one, of a number or list of elements. Only terms clearly indicated to the contrary, such as “only one of or “exactly one of,” will refer to the inclusion of exactly one element of a number or list of elements.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1.-68. (canceled)

69. A microfluidic device for at least one of capturing, sorting, analyzing, typing and cultivating analytes, comprising at least a microchannel comprising at least an active zone, said active zone comprising at least a capture element.

70. A microfluidic device according to claim 69, wherein the width of said active zone, or the combined width of active zones, perpendicular to the direction of flow, is larger than their effective length in the direction of the flow.

71. A microfluidic device according to claim 69, wherein said at least one capture element is activable.

72. A microfluidic device according to claim 69, wherein said at least one capture element is magnetic.

73. A microfluidic device according to claim 69, wherein said at least one active zone is closed on at least one of its sides by a transparent window, with a thickness smaller than 500 μm.

74. A microfluidic device according to claim 69; wherein the thickness of said active zone is comprised on at least part of its surface between 20 μm and 100 μm.

75. A microfluidic device according to claim 69, wherein the combined thickness of said window and said active zone is, on at least part of the area of said window, smaller than 300 μm.

76. A microfluidic device according to claim 69, wherein said capture elements have a size comprised between 10 nm and 50 nm, or between 50 nm and 200 nm, or between 200 nm and 500 nm, or between 500 nm and 1 μm, or between 1 μm and 2 μm, or between 2 μm and 5 μm, or between 5 μm and 10 μm, or between 10 μm and 20 μm, or between 20 μm and 50 μm.

77. A microfluidic device according to claim 69, comprising in addition a second analysis zone, and means to transport the analytes from the active zone to the analysis zone.

78. A method for at least one of the sorting, screening, study, storage and culture of analytes wherein said analytes are flowed across a microfluidic device according to claim 69.

79. A method according to claim 78, comprising in addition at least one of:
rinsing at least part of said microfluidic system with a fluid containing no capture colloidal objects able to assemble onto said capture elements, and no analytes,
flowing reagents into said at least one active zone whereas said first means are kept activated,
flowing into said active area a mounting agent, or a hardenable material,
moving said microfluidic device from a first instrument in which the capture of analytes is performed, to a second instrument in which the analysis or imaging of analytes is performed.

80. A method according to claim 79, wherein said reagents comprise at least one type of reagents for revealing biomarkers.

81. A method according to claim 78, comprising performing immunophenotyping of at least one of captured analytes within said active zone.

82. A method according to claim 78, comprising analysing nucleic acid sequences in at least one of captured analytes.

83. A method according to claim 78, wherein said method is used for screening a drug, chemical or compound for its toxicity, efficiency or biological effect.

84. A method according to claim 78, wherein at least two populations of beads with well distinct sizes or well distinct magnetization are flowed in a microchannel comprising at least an active zone, said active zone comprising at least a capture element,
at least one of said two populations of beads being flowed in said microchannel in the absence of said analytes, and at least one of said populations of beads carrying ligands for said analytes.

85. A method according to claim 78, comprising in addition a step of releasing analytes from said active zone, and a step of at least one of analyzing, cultivating, and differentiating said analytes in at least one second analysis zone.

86. A method according to claim 78, comprising a first step of providing a first blood sample of volume A, and at least a second step of flowing said sample or a pretreated sample obtained from said first blood sample in an active zone or a combination of active zones, where said analytes are captured,
wherein said flowing step lasts less than two hours, and wherein in less than 1 hour, with a ratio between the initial sample volume A to the volume of the active zone of a microfluidic device for at least one of capturing, sorting, analyzing, typing and cultivating analytes or the combined volume of the active zones of said microfluidic device in which said cells are captured is larger than 100.

87. A method for magnetic capture of cells or analytes from an initial raw sample, with a microfluidic device, comprising treating at least 1 mL of raw sample by using magnetic particles having a total mass of less that 10 mg.

88. A method according to claim 78, wherein the sample containing said analytes is treated by a pretreatment method that does not involve either lysis or Ficoll, or by a pretreatment method suitable to dissociate and suspend cells in a liquid medium, prior to flowing said analytes in said microfluidic device.

89. A method according to claim 87, wherein the sample containing said analytes is treated by a pretreatment method that does not involve either lysis or Ficoll, or by a pretreatment method suitable to dissociate and suspend cells in a liquid medium, prior to flowing said analytes in said microfluidic device.
